(19) United States
(12) Patent Application Publication
(10) Pub. No.: US 2023/0107167 A1
(43) Pub. Date: Apr. 6, 2023

(54) VISION SCREENING DEVICE, VISION IMPAIRMENT SCREENING SYSTEM, IMPROVED VISION SCREENING PROCESS, METHOD OF ASSESSMENT, AND METHOD OF USE

(71) Applicant: Axiom Innova, Inc., Reno, NV (US)

(72) Inventors: Michael Brownell, Reno, NV (US); Patrick DeGuzman, Reno, NV (US); Darcy Wendel, Reno, NV (US); David Huang, Reno, NV (US)

(73) Assignee: Axiom Innova, Inc., Reno, NV (US)

(21) Appl. No.: 17/493,421

(22) Filed: Oct. 4, 2021

(51) Int. Cl. A61B 3/113; A61B 3/12; A61B 3/00

(52) U.S. Cl. CPC A61B 3/113; A61B 3/12; A61B 3/0058; A61B 3/005; A61B 3/0008

(57) ABSTRACT

A vision screening device, a vision screening system, a vision impairment screening device, vision impairment screening system, processes, method of assessments, and methods of use are presented. The present disclosure provides a vision screening system and device, including obtaining flash imagery in order to be used for analysis of visual function. More specifically, and without limitation, the present disclosure provides the state of the art with a system for attracting the gaze of a user and controlling accommodation for obtaining proper imagery as is needed for visual analysis. Furthermore, and without limitation, the present disclosure provides a vision system for capturing visual performance of a user.

### FIELD OF THE DISCLOSURE

This disclosure relates to a vision screening device, a vision screening system, a vision impairment screening device, vision impairment screening system, processes, method of assessments, and methods of use. More specifically, and without limitation, the present disclosure relates to a vision screening system for assessment of visual function. Furthermore, and without limitation, the present disclosure relates to a vision impairment screening system for detection of risk factors for vision impairment and more.

### COPYRIGHT NOTICE

At least a portion of the disclosure of this patent document contains material that is subject to copyright protection. The copyright owner has no objection to the facsimile reproduction by anyone of the patent document or the patent disclosure, as it appears in the Patent and Trademark Office patent files and/or records, but otherwise reserves all copyright rights whatsoever. The following notice applies to the software and data as described below and in the drawings that form a part of this document. Copyright Gobiquity. All rights reserved.

### BACKGROUND OF THE DISCLOSURE

Vision care includes screening, diagnosing, and treating visual disorders and sight threatening conditions. Vision care providers can include, but are not limited to, ophthalmologists, optometrists, primary care providers, and pediatricians. A vision care provider performs a series of examinations and/or tests on the client's vision to determine needs and a course of action, as required. These examinations involve examining the patient's eyes and visual performance for risk factors, defects, or abnormalities, as well as, potentially taking actions, such as, prescribing corrective lenses and/or other eye treatments and/or therapies.

Early intervention in any eye treatment or eye disease can often lead to vision saving results. Furthermore, catching vision impairment can also lead to the creation of visual aids such as lenses or eyeglasses or contacts to help a patient see better. It is through comprehensive eye examinations which take place at an eye care provider's office, community setting, or remotely, by which these treatments and solutions are developed.

Typically, an eye care professional will diagnose and help a patient manage any vision risks or abnormalities, including refractive error and visual impairment, as well as more significant eye diseases. Early and effective vision screening is an important factor in providing quality eye health care, and can reduce risk of vision loss and avoid expensive treatments at a later stage. These treatments can be expensive, especially if a patient doesn't have any kind of health insurance or health financial coverage.

Availability of quality vision screening in pediatrics and primary care can be a problem due to lack of vision testing equipment, and these are the providers that see most of the general population. Furthermore, in order to see specialists for any required vision treatments, a patient must get a referral from a physician, typically in primary care, pediatrics, or optometry. So there is a critical need for effective, easy to use, and broadly available vision screening and testing capabilities, such as low cost mobile platforms. This also enables remote use in geographically isolated regions, at schools and community locations, or when visiting an office is difficult due to healthcare concerns like a pandemic or illness.

While effective screening for vision health is critical in adults, it is also critical in children. Children may not be able to effectively communicate some of their vision needs. Furthermore, vision is an important overall health aspect of human health.

As one example, amblyopia is the leading cause of vision impairment in children. Vision impairment in children will affect that child for the rest of their lives, and it will greatly impact the ability to learn and socialize, which are key skills for success. Early screening of this condition provides the best opportunity to prevent vision impairment and correct a child's vision.

Thus, there is a long-felt need in the art for a novel and easy vision screening process for detection of risk factors and prevention of vision impairment. Furthermore, there is a long-felt need in the art for a reliable, easy to use vision screening system which can be readily available and used anywhere at a user's convenience.

This goal has many complications, which have been plaguing the art, from the performance of a photographic screening systems, to obtaining needed environmental conditions for the screening process to be effective. Furthermore, the gaze of a user must be in certain directions and for certain durations of time to get an accurate measurement, and the focus of the patient's eyes must be in a particular direction and at a particular distance for effective screening. Many other complications also plague the art of making a widely available and easy to use vision screening system.

The disclosure herein provides these advantages, solves these problems and others, as will become clear from the specification and claims provided.

### SUMMARY OF THE DISCLOSURE

A vision screening device, a vision screening system, a vision impairment screening device, vision impairment screening system, processes, method of assessments, and methods of use are presented. More specifically, and without limitation, the present disclosure relates to a vision screening system for assessment of visual function. Furthermore, and without limitation, the present disclosure relates to a vision impairment screening system for detection of risk factors for vision impairment and more.

Furthermore, and without limitation, the present disclosure relates to a vision screening system and device, including obtaining flash imagery in order to be used for analysis of visual function. More specifically, and without limitation, the present disclosure provides the state of the art with a system for attracting the gaze of a user for obtaining proper imagery as is needed for visual analysis. Furthermore, and without limitation, the present disclosure relates to a vision system for capturing visual performance of a subject.

In the arrangement shown, as one example, the vision screening system presented utilizes a mobile device, smart device, tablet, smartphone, and the like for vision screening. This includes, but is not limited to eccentric photoscreening and other methods disclosed herein. The present system is configured to acquire information related to the key features of an eye and image of an eye including dimensional ratios and other information that are evaluated and compared by the system and the established risk criteria and other predetermined rules and parameters.

In the arrangement shown, as one example, the present disclosure utilizes a strategically located light source near the photoscreening camera to attract the attention of a patient so that the patient looks in the desired direction at the time imagery is acquired. In this way, the present disclosure improves upon the accuracy, measurements, and usability of images acquired from the user's gaze. Furthermore, in the present disclosure the lighting intentionally influences the focus of the eye in order to minimize visual accommodation and improve on the imagery collected, which affects the measurements of the eye.

Said another way, for many vision screening and eye examination methods, it is preferred that the subject's eyes be focused at infinity, or far into the distance. Furthermore, when the eye changes focus from near to intermediate to far, the eye is known to accommodate and the effective power of the eye is changing. The optical power is just one parameter the present disclosure is configured to measure. Furthermore, the present disclosure uses these and other parameters to assess risk factors.

In the arrangement shown, as one example, the lighting feature design (further described herein) defocuses the illuminated images provided. This feature attracts the gaze of a child and/or adult directly to the camera while causing the user's eye to focus at “infinity” and on a central element of the present disclosure.

In this way, the present disclosure optimizes the results of a vision test by providing features which cause the eyes of an adult and/or child to focus on an object at a far distance in the direction of the camera, and ensures the illumination and image capture are optimized. So, in this way, presenting these features of the present disclosure with an image that is blurry will cause the eye to focus at infinity and/or cause the focus of a user's eyes with minimal gaze deviation, and enables superior image acquisition for analysis of vision. Furthermore, the sensors and other functionality of the present disclosure improve usability and performance. Other sensors, further described herein, include but are not limited to distance sensors, illumination augmentation, and other capture and measurement capabilities and functionality.

In the arrangement shown, as one example, the present disclosure provides a defocused, illuminated image. In one arrangement, the defocused, illuminated image is a blurred, light emitting diode light array. In one arrangement, and as will be discussed further herein, the blurred, light emitting diode light array is situated adjacent and/or near the camera and the flash of the device. This device, as disclosed herein, is referred to as an attractor.

In the arrangement shown, as one example, the attractor is configured as various images, various designs, dynamic designs, colors, etc. In this way, the present disclosure also includes a module for the attractor display with various electronic components, including but not limited to a power source. In one arrangement, the power source is configured to attach to a phone case.

In the arrangement shown, as one example, the brightness of the illuminated image, the amount of focus and/or the attractiveness of the image—including but not limited to color, shape, dynamics, blinking, dimensional changes—are important factors which aid in the efficacy of the attractor and usability in a particular screening environment. These elements are programmed by a predetermined set of rules but are also adjustable in the present disclosure through the user interface and various control elements such as the attractor module.

Furthermore, and in the arrangement shown, the attractor module may also include rechargeable batteries, power indicators, a power switch, various sensors, image capture elements, and various measurement sensors. Furthermore, and in an alternative embodiment, the distant sensors are configured to provide proper vision screening distance. Furthermore, and in an alternative embodiment, the flash brightness is augmented using a concentrating lens and/or flash enhancer module. In this way, the brightness of the existing flash on a mobile device can be improved through the configuration provided herein to improve the illumination of the patient's eye for visual screening performance and usability improvements.

Thus, the present disclosure provides for a vision screening device, a vision screening system, a vision impairment screening device, vision impairment screening system, processes, method of assessments, and methods of use that provides the state of the art with a novel, convenient vision screening system and vision impairment detection system which is convenient, can be used at home, and more.

Thus, the present disclosure provides a vision screening system which includes, but is not limited to, attracting the proper gaze of a user for the proper duration of time, and in the proper direction and focus. Furthermore, the present disclosure provides for minimization of visual accommodation. Said another way, the present disclosure provides for minimizing the refocusing of the eye at different distances. Furthermore, the present disclosure provides for a flash and camera which can provide for effective photoscreening and includes a plurality of sensors which evaluate distance and other parameters. Furthermore, the plurality of sensors, further described herein, capture images and other test data which can be processed in the external models (further described herein) for vision screening. Furthermore, the present disclosure provides usability by minimizing retesting, image retakes, providing proper gaze, proper distance, proper illumination, required image quality, and other factors. These and other factors further described herein provide for a reliable and robust vision screening system which is easily accessible.

Thus, it is a primary object of the disclosure to provide a vision screening device, a vision screening system, a vision impairment screening device, vision impairment screening system, processes, method of assessments, and methods of use that improves upon the state of the art.

Another object of the disclosure is to provide a vision screening device, a vision screening system, a vision impairment screening device, vision impairment screening system, processes, method of assessments, and methods of use that provides a portable vision screening device.

Yet another object of the disclosure is to provide a vision screening device, a vision screening system, a vision impairment screening device, vision impairment screening system, processes, method of assessments, and methods of use that integrate with smartphones.

Another object of the disclosure is to provide a vision screening device, a vision screening system, a vision impairment screening device, vision impairment screening system, processes, method of assessments, and methods of use that integrate with smart phone flash photography features.

Yet another object of the disclosure is to provide a vision screening device, a vision screening system, a vision impairment screening device, vision impairment screening system, processes, method of assessments, and methods of use that detect risk factors associated with eye diseases and issues.

Another object of the disclosure is to provide a vision screening device, a vision screening system, a vision impairment screening device, vision impairment screening system, processes, method of assessments, and methods of use that effectively evaluates key features of an eye.

Yet another object of the disclosure is to provide a vision screening device, a vision screening system, a vision impairment screening device, vision impairment screening system, processes, method of assessments, and methods of use that effectively evaluates dimensional ratios.

Another object of the disclosure is to provide a vision screening device, a vision screening system, a vision impairment screening device, vision impairment screening system, processes, method of assessments, and methods of use that effectively evaluates an eye against established risk criteria.

Yet another object of the disclosure is to provide a vision screening device, a vision screening system, a vision impairment screening device, vision impairment screening system, processes, method of assessments, and methods of use that provides an additional light source.

Another object of the disclosure is to provide a vision screening device, a vision screening system, a vision impairment screening device, vision impairment screening system, processes, method of assessments, and methods of use that provides a camera for photoscreening.

Yet another object of the disclosure is to provide a vision screening device, a vision screening system, a vision impairment screening device, vision impairment screening system, processes, method of assessments, and methods of use that provides a light source to attract the attention of a patient.

Another object of the disclosure is to provide a vision screening device, a vision screening system, a vision impairment screening device, vision impairment screening system, processes, method of assessments, and methods of use that provides a light source which causes a patient to look in a desired direction and/or at a desired angle.

Yet another object of the disclosure is to provide a vision screening device, a vision screening system, a vision impairment screening device, vision impairment screening system, processes, method of assessments, and methods of use that provides a light source which causes a patient to have a desired gaze.

Another object of the disclosure is to provide a vision screening device, a vision screening system, a vision impairment screening device, vision impairment screening system, processes, method of assessments, and methods of use that provides a source which causes a patient to have a desired eye focus.

Yet another object of the disclosure is to provide a vision screening device, a vision screening system, a vision impairment screening device, vision impairment screening system, processes, method of assessments, and methods of use that causes a patient to focus at a far distance.

Another object of the disclosure is to provide a vision screening device, a vision screening system, a vision impairment screening device, vision impairment screening system, processes, method of assessments, and methods of use that causes a patient to focus at a near distance.

Yet another object of the disclosure is to provide a vision screening device, a vision screening system, a vision impairment screening device, vision impairment screening system, processes, method of assessments, and methods of use that causes a patient to focus on a medium distance.

Another object of the disclosure is to provide a vision screening device, a vision screening system, a vision impairment screening device, vision impairment screening system, processes, method of assessments, and methods of use that provides a plurality of sensor types.

Yet another object of the disclosure is to provide a vision screening device, a vision screening system, a vision impairment screening device, vision impairment screening system, processes, method of assessments, and methods of use that provides illumination augmentation.

Another object of the disclosure is to provide a vision screening device, a vision screening system, a vision impairment screening device, vision impairment screening system, processes, method of assessments, and methods of use that provides for analysis of images captured based on predetermined rules.

Yet another object of the disclosure is to provide a vision screening device, a vision screening system, a vision impairment screening device, vision impairment screening system, processes, method of assessments, and methods of use that provide an attractor module.

Another object of the disclosure is to provide a vision screening device, a vision screening system, a vision impairment screening device, vision impairment screening system, processes, method of assessments, and methods of use that provides instant screening results.

Yet another object of the disclosure is to provide a vision screening device, a vision screening system, a vision impairment screening device, vision impairment screening system, processes, method of assessments, and methods of use that can track historical data.

Another object of the disclosure is to provide a vision screening device, a vision screening system, a vision impairment screening device, vision impairment screening system, processes, method of assessments, and methods of use that provides alerts.

Yet another object of the disclosure is to provide a vision screening device, a vision screening system, a vision impairment screening device, vision impairment screening system, processes, method of assessments, and methods of use that are easy to use.

Another object of the disclosure is to provide a vision screening device, a vision screening system, a vision impairment screening device, vision impairment screening system, processes, method of assessments, and methods of use that are mobile.

Yet another object of the disclosure is to provide a vision screening device, a vision screening system, a vision impairment screening device, vision impairment screening system, processes, method of assessments, and methods of use that are safe to use.

Another object of the disclosure is to provide a vision screening device, a vision screening system, a vision impairment screening device, vision impairment screening system, processes, method of assessments, and methods of use that are accurate.

Yet another object of the disclosure is to provide a vision screening device, a vision screening system, a vision impairment screening device, vision impairment screening system, processes, method of assessments, and methods of use that are robust.

Another object of the disclosure is to provide a vision screening device, a vision screening system, a vision impairment screening device, vision impairment screening system, processes, method of assessments, and methods of use that integrate with various digital platforms.

Yet another object of the disclosure is to provide a vision screening device, a vision screening system, a vision impairment screening device, vision impairment screening system, processes, method of assessments, and methods of use that provides a user interface.

Another object of the disclosure is to provide a vision screening device, a vision screening system, a vision impairment screening device, vision impairment screening system, processes, method of assessments, and methods of use that are quick.

Yet another object of the disclosure is to provide a vision screening device, a vision screening system, a vision impairment screening device, vision impairment screening system, processes, method of assessments, and methods of use that are efficient.

Another object of the disclosure is to provide a vision screening device, a vision screening system, a vision impairment screening device, vision impairment screening system, processes, method of assessments, and methods of use that are programmed with various predetermined rule sets.

Yet another object of the disclosure is to provide a vision screening device, a vision screening system, a vision impairment screening device, vision impairment screening system, processes, method of assessments, and methods of use that integrate machine learning.

Another object of the disclosure is to provide a vision screening device, a vision screening system, a vision impairment screening device, vision impairment screening system, processes, method of assessments, and methods of use that integrate and/or are programmable for the latest eye knowledge.

Yet another object of the disclosure is to provide a vision screening device, a vision screening system, a vision impairment screening device, vision impairment screening system, processes, method of assessments, and methods of use that are high quality.

Another object of the disclosure is to provide a vision screening device, a vision screening system, a vision impairment screening device, vision impairment screening system, processes, method of assessments, and methods of use that save time for a user.

These and other objects, features, or advantages of the present disclosure will become apparent from the specification and claims.

### BRIEF DESCRIPTION OF THE DRAWINGS

FIG. 1 is a top, perspective view showing the attractor module; the view showing the system; the view showing an attractor; the view showing a housing; the view showing a diffuser.

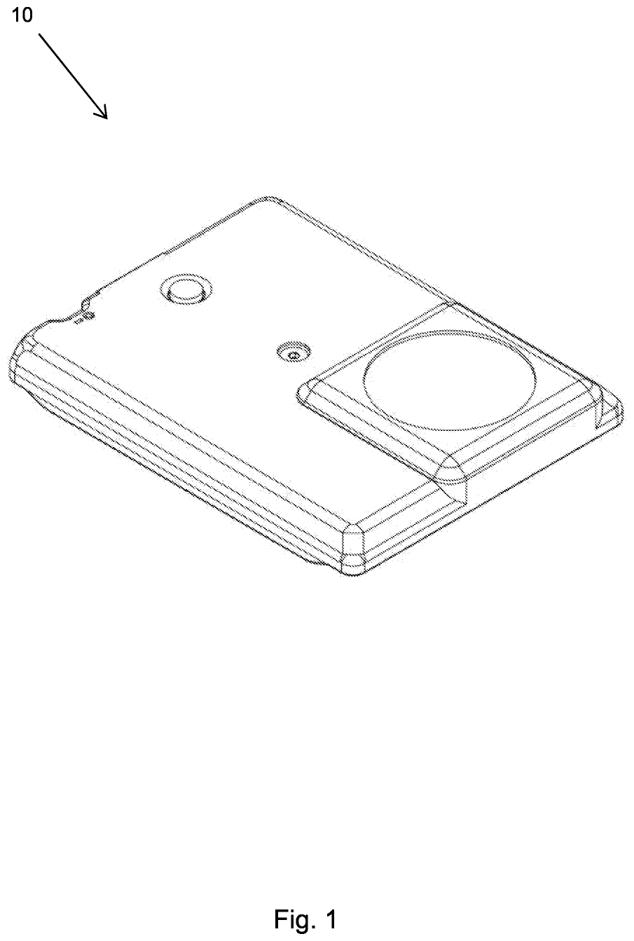

FIG. 2 is a top, perspective view showing the attractor module; the view showing the system; the view showing an attractor; the view showing a housing; the view showing a diffuser; the view showing a diffuser housing; the view showing a diffuser liner; the view showing a plurality of connection features.

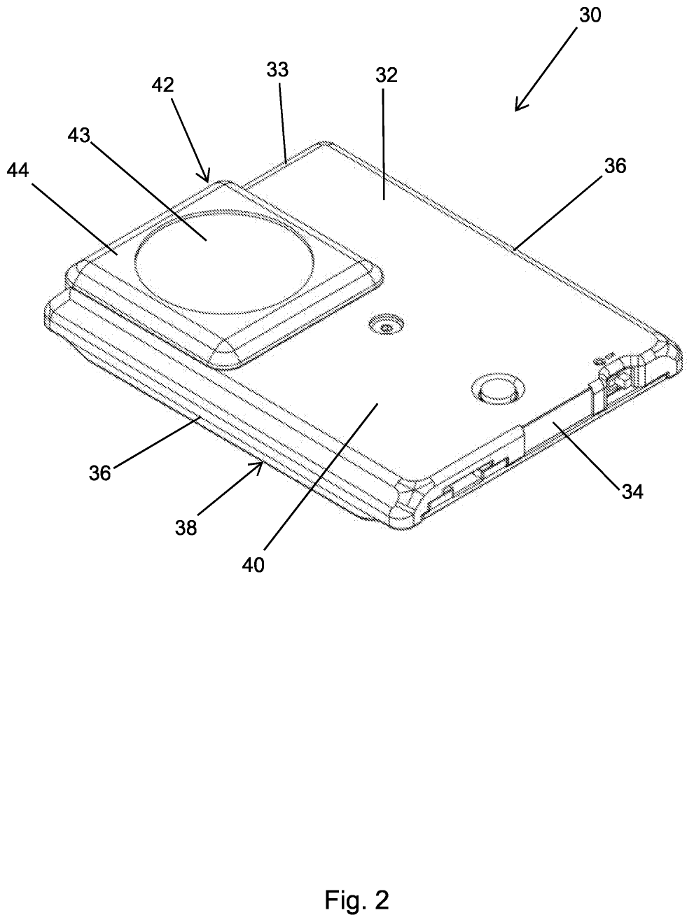

FIG. 3 is a front view showing the attractor module; the view showing the system; the view showing an attractor; the view showing a housing; the view showing a diffuser; the view showing a diffuser housing; the view showing a diffuser liner; the view showing a plurality of connection features.

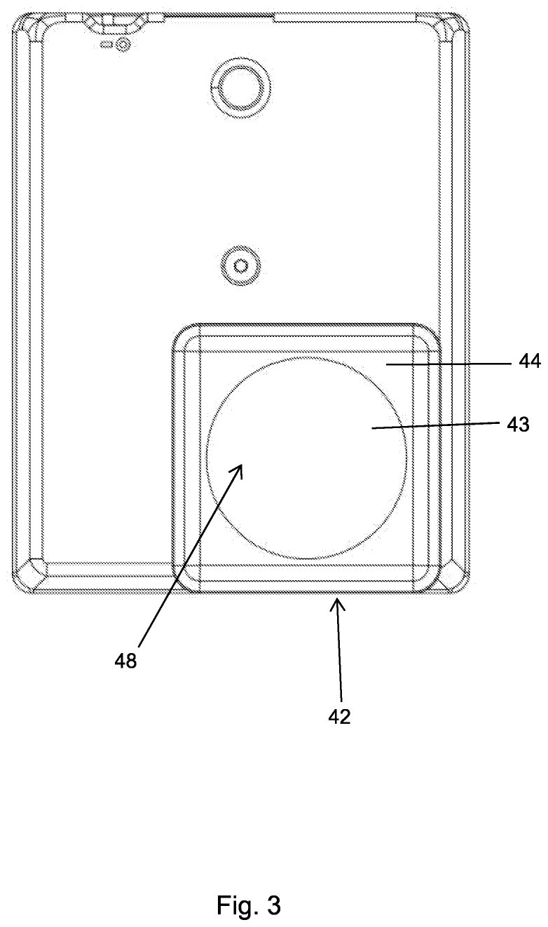

FIG. 4 is a bottom view showing the attractor module; the view showing the system; the view showing an attractor; the view showing a housing; the view showing the first end of the housing; the view showing a plurality of attachment features.

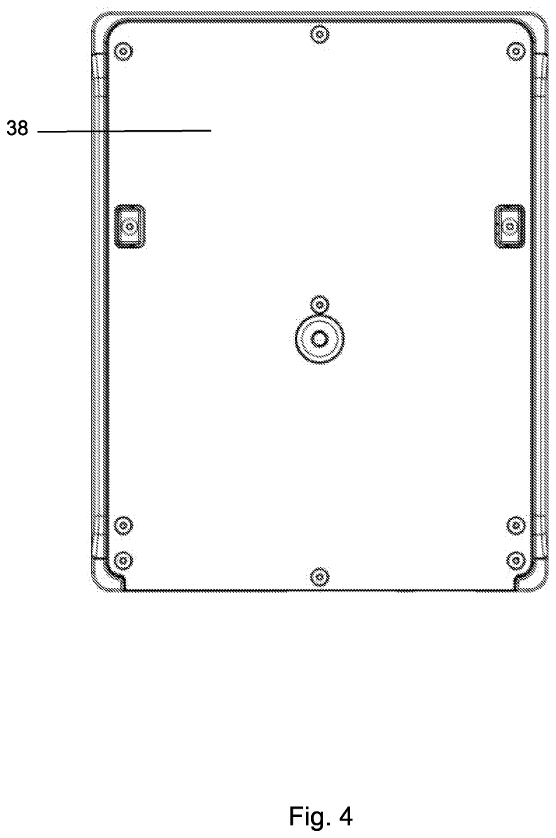

FIG. 5 is a top, elevation view showing the attractor module; the view showing the system; the view showing an attractor; the view showing a housing; the view showing a diffuser housing; the view showing a plurality of attachment features.

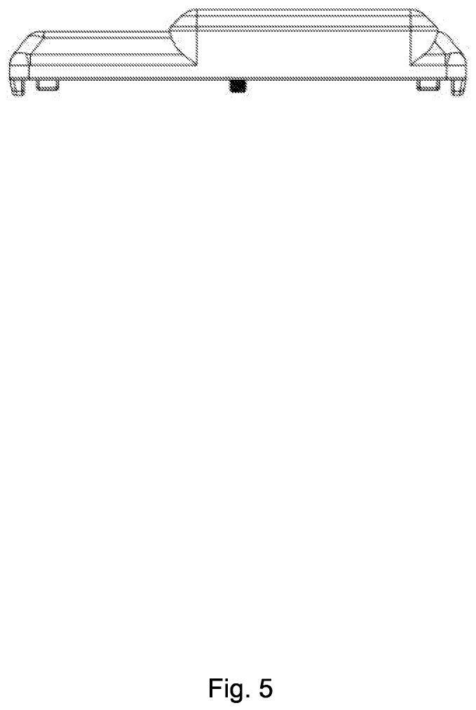

FIG. 6 is a bottom, elevation view showing the attractor module; the view showing the system; the view showing an attractor; the view showing a housing; the view showing a diffuser housing; the view showing a plurality of connection features; the view showing a plurality of attachment features.

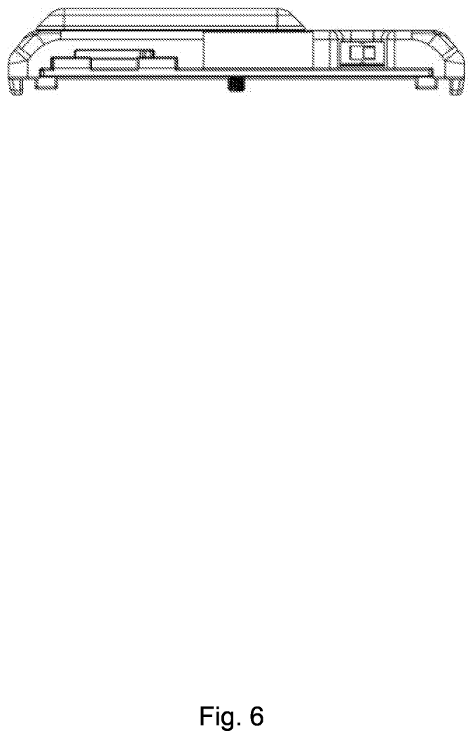

FIG. 7 is a side elevation view showing the attractor module; the view showing the system; the view showing an attractor; the view showing a housing; the view showing a diffuser housing; the view showing the opposing sides.

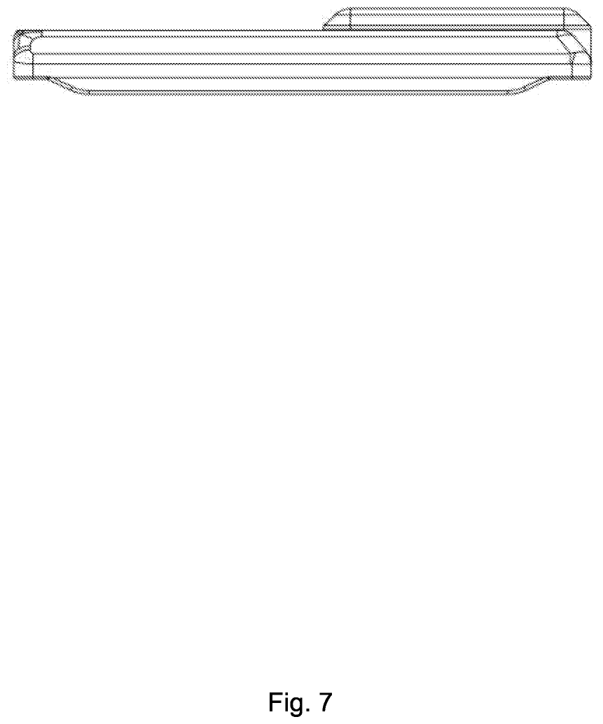

FIG. 8 is a side elevation view showing the attractor module; the view showing the system; the view showing an attractor; the view showing a housing; the view showing a diffuser housing; the view showing the opposing sides.

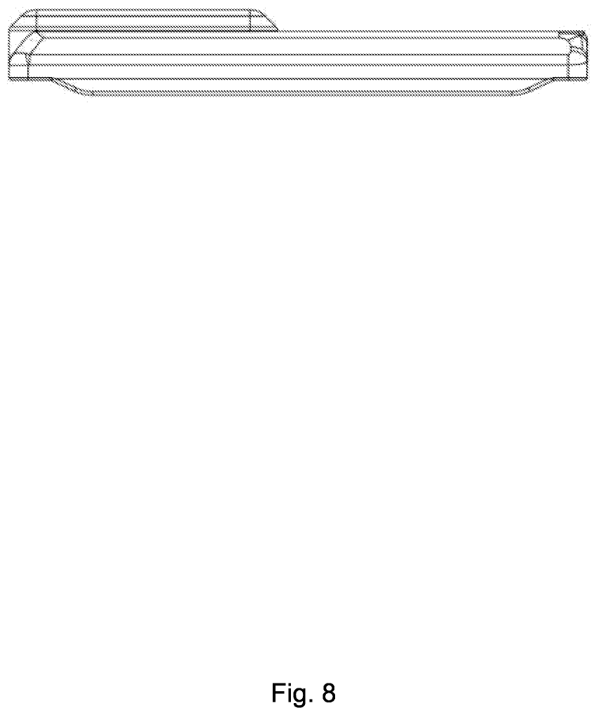

FIG. 9 is a bottom, perspective view showing the attractor module; the view showing the system; the view showing an attractor; the view showing a housing; the view showing a plurality of attachment features.

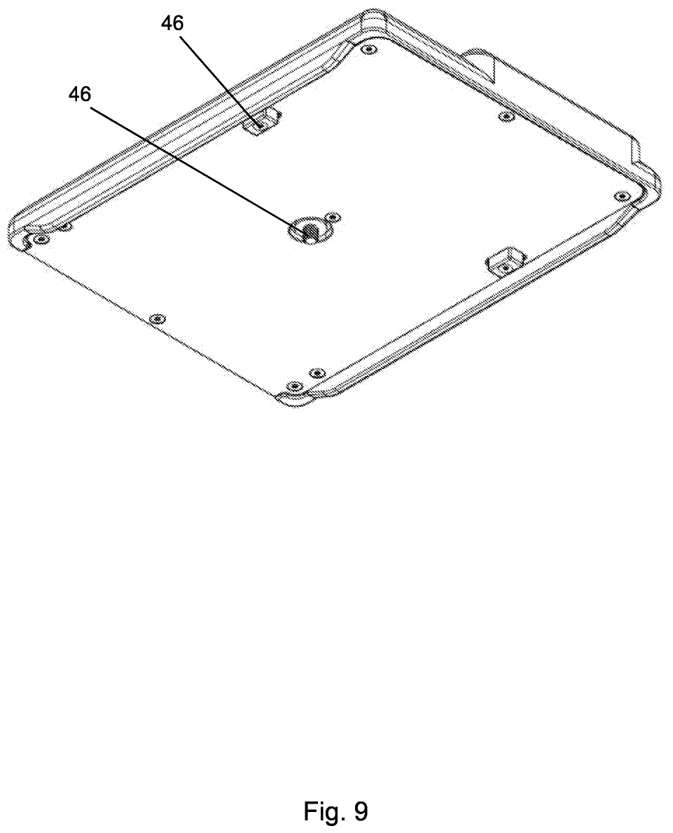

FIG. 10 is a bottom, perspective view showing the attractor module; the view showing the system; the view showing an attractor; the view showing a housing; the view showing a plurality of attachment features.

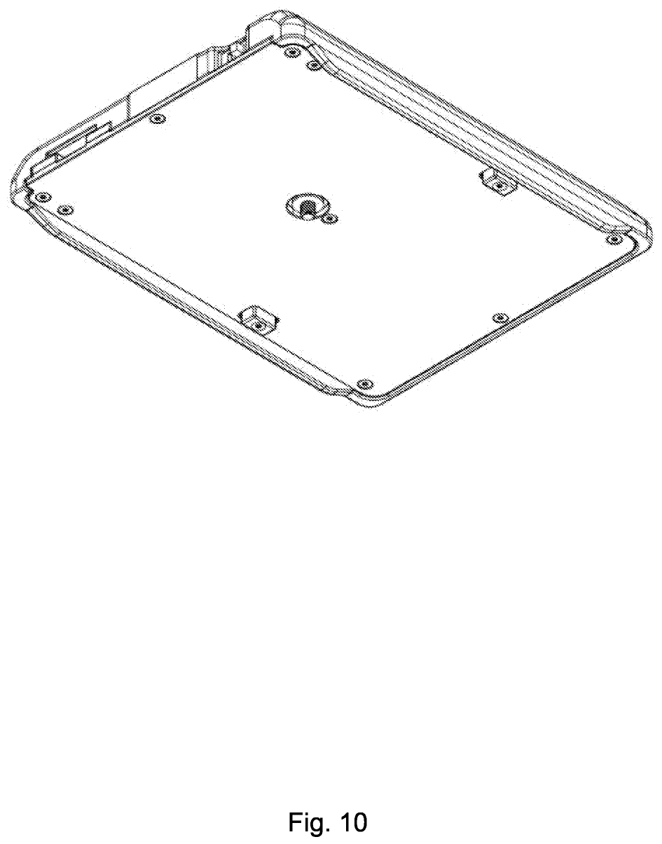

FIG. 11 is an exploded isometric view showing the attractor module; the view showing the system; the view showing an attractor; the view showing a housing; the view showing a diffuser; the view showing a diffuser housing; the view showing a diffuser liner; the view showing a plurality of connection features; the view showing onboard computing components; the view showing an on/off switch.

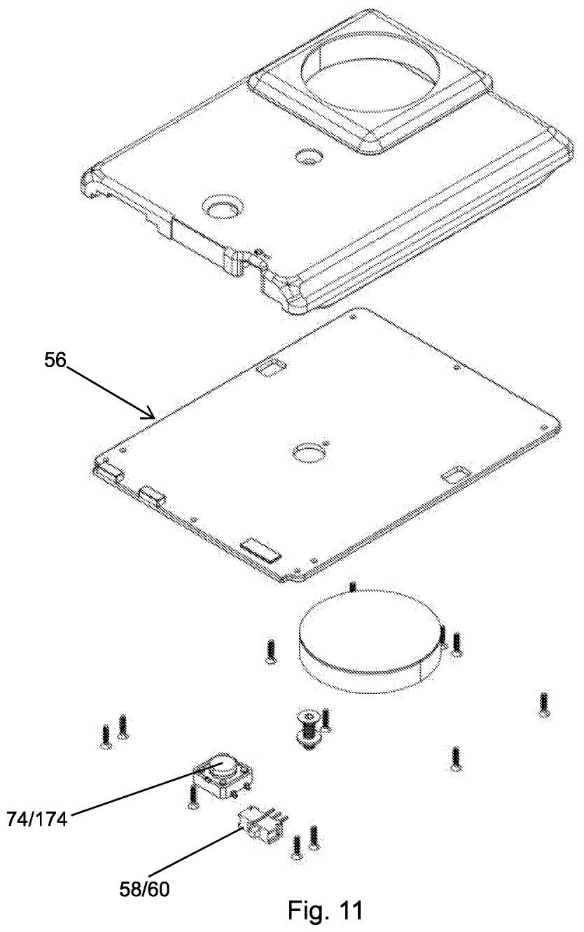

FIG. 12 is an exploded isometric view showing the attractor module; the view showing the system; the view showing an attractor; the view showing a housing; the view showing a diffuser; the view showing a diffuser housing; the view showing a diffuser liner; the view showing a plurality of connection features; the view showing onboard computing components; the view showing an on/off switch.

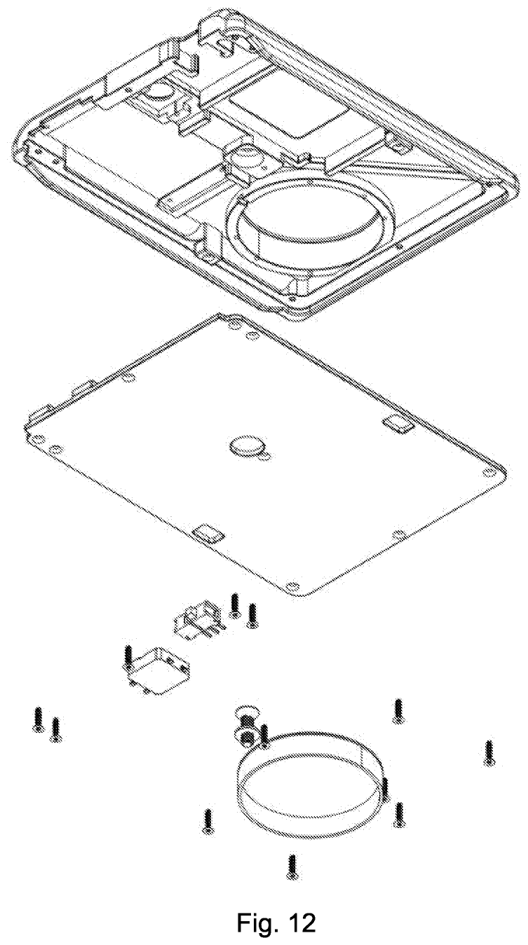

FIG. 13 is a top, perspective view showing the case attractor module embodiment; the view showing a smart device; the view showing the graphical user interface representation and display of the smart device; the view showing the smart device attached to the case attractor module.

FIG. 14 is a top, perspective view showing the case attractor module embodiment; the view showing a smart device; the view showing the graphical user interface representation and display of the smart device; the view showing the smart device attached to the case attractor module.

FIG. 15 is a front view showing the case attractor module embodiment; the view showing a smart device; the view showing the graphical user interface representation and display of the smart device; the view showing the smart device attached to the case attractor module; the view showing the housing; the view showing the top of the housing; the view showing the bottom of the housing; the view showing the opposing sides of the housing.

FIG. 16 is a rear view showing the case attractor module embodiment; the view showing a smart device; the view showing the graphical user interface representation and display of the smart device; the view showing the smart device attached to the case attractor module; the view showing the diffuser; the view showing the diffuser housing; the view showing a plurality of attachment features; the view showing a camera filter; the view showing a flash.

FIG. 17 is a top view showing the case attractor module embodiment; the view showing a smart device; the view showing the diffuser; the view showing the diffuser housing; the view showing a plurality of attachment features; the view showing a camera filter; the view showing a flash filter.

FIG. 18 is a bottom view showing the case attractor module embodiment; the view showing a smart device; the view showing the diffuser; the view showing the diffuser housing; the view showing a plurality of attachment features; the view showing a camera filter; the view showing a flash filter; the view showing a plurality of apertures; the view showing a smart device.

FIG. 19 is a side elevation view showing the case attractor module embodiment; the view showing a smart device; the view showing the smart device attached to the case attractor module; the view showing the diffuser; the view showing the diffuser housing; the view showing a plurality of attachment features; the view showing a camera filter; the view showing a flash filter.

FIG. 20 is a side elevation view showing the case attractor module embodiment; the view showing a smart device; the view showing the smart device attached to the case attractor module; the view showing the diffuser; the view showing the diffuser housing; the view showing a plurality of attachment features; the view showing a camera filter; the view showing a flash filter.

FIG. 21 is a bottom perspective view showing the case attractor module embodiment; the view showing a smart device; the view showing the smart device attached to the case attractor module; the view showing the diffuser; the view showing the diffuser housing; the view showing a plurality of attachment features; the view showing a camera filter; the view showing a flash filter.

FIG. 22 is a bottom perspective view showing the case attractor module embodiment; the view showing a smart device; the view showing the smart device attached to the case attractor module; the view showing the diffuser; the view showing the diffuser housing; the view showing a plurality of attachment features; the view showing a camera filter; the view showing a flash filter.

FIG. 23 is an exploded isometric view showing the case attractor module embodiment; the view showing a smart device; the view showing the smart device attached to the case attractor module; the view showing the diffuser; the view showing the diffuser housing; the view showing a plurality of attachment features; the view showing a camera filter; the view showing a flash filter.

FIG. 24 is an exploded isometric view showing the case attractor module embodiment; the view showing a smart device; the view showing the smart device attached to the case attractor module; the view showing the diffuser; the view showing the diffuser housing; the view showing a plurality of attachment features; the view showing a camera filter; the view showing a flash filter.

### DETAILED DESCRIPTION OF THE DISCLOSURE

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the disclosure(s). The following detailed description is, therefore, not to be taken in a limiting sense, and the scope of the disclosure(s) is defined only by the appended claims, along with the full scope of equivalents to which such claims are entitled.

As used herein, the terminology such as vertical, horizontal, top, bottom, front, back, end, sides and the like are referenced according to the views, pieces and figures presented. It should be understood, however, that the terms are used only for purposes of description, and are not intended to be used as limitations. Accordingly, orientation of an object or a combination of objects may change without departing from the scope of the disclosure.

Reference throughout this specification to “one embodiment,” “an embodiment,” “one example,” or “an example” means that a particular feature, structure, or characteristic described in connection with the embodiment or example is included in at least one embodiment of the present disclosure. Thus, the appearance of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Embodiments in accordance with the present disclosure may be embodied as an apparatus, method, or computer program product. Accordingly, the present disclosure may take the form of an entirely hardware-comprised embodiment, an entirely software-comprised embodiment (including firmware, resident software, micro-code, etc.), or an embodiment combining software and hardware aspects that may all generally be referred to herein as a “circuit,” “module,” or “system.” Furthermore, embodiments of the present disclosure may take the form of a computer program product embodied in any tangible medium.

Any combination of one or more computer-usable or computer-readable media may be utilized. For example, a computer-readable medium may include one or more of a portable computer removable drive, a hard disk, a random access memory (RAM) device, a read-only memory (ROM) device, an erasable programmable read-only memory (EPROM or Flash memory) device, a portable compact disc read-only memory (CDROM), an optical storage device, and a magnetic storage device. Computer program code for carrying out operations of the present disclosure may be written in any combination of one or more programming languages. Such code may be compiled from source code to computer-readable assembly language or machine code, or virtual code, or framework code suitable for the disclosure herein, or machine code suitable for the device or computer on which the code will be executed.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction and then scaled accordingly. A cloud model can be composed of various characteristics (e.g., on-demand self-service, broad network access, resource pooling, rapid elasticity, and measured service), service models (e.g., Software as a Service (“Saas”), Platform as a Service (“PaaS”), and Infrastructure as a Service (“IaaS”)), and deployment models (e.g., private cloud, community cloud, public cloud, and hybrid cloud).

Any flowcharts and block diagrams in the attached figures illustrate the architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart and/or block diagram block or blocks.

In the arrangement shown, as one example, a vision screening system for the assessment of visual capability and risk factors for vision impairment for an individual is presented. In the arrangement shown, as one example, the disclosure provides the system for correctly obtaining a flash photograph of a patient's eye under conditions necessary for analysis of the visual function of a user's eye.

Furthermore, and in the arrangement shown, as one example, the present disclosure provides the state of the art with an attraction feature for attracting the gaze of a user, such as a child, during the photo screening and/or photo obtaining process. In this way, the attractor feature of the present disclosure provides and/or improves upon image capture functionality of the state of the art. Said another way, the present disclosure provides and/or improves upon capturing images of the eye which can be used for analysis of vision function and/or the analysis of vision impairment and/or the analysis of vision performance.

In the arrangement shown, as one example, an easy to use and convenient vision screening and assessment system and instruments are presented. In this way, the present disclosure provides the state of the art with an effective system for vision screening and photo screening. Said another way, the present disclosure provides a system and modules for capturing images for photo screening and evaluating these images.

Furthermore, and in the arrangement shown, as one example, the present disclosure provides improved vision screening and testing. The present disclosure provides improved screening by minimizing retesting and/or recapturing images and test data due to improper gaze, distance a subject is from the system, flash concentration, image qualification, and other factors.

Some of this data capture includes, but is not limited to capturing images, object recognition within images, counting of objects within images, combined with location, spatial geometries, historical data including location of regular anomalies, and other information can be used to create customized information, identify patterns, identifying eye diseases, identifying potential risks or problems, identifying vision impairment and other issues, and more.

### System

With reference to the figures, a vision screening device, a vision screening system, a vision impairment screening device, vision impairment screening system, processes, method of assessments, and methods of use 10 are presented. Vision screening device, vision screening system, vision impairment screening device, vision impairment screening system, processes, method of assessments, and methods of use 10 (hereafter referred to as “vision screening system”, “photo screening system”, “vision impairment screening system”, “screening system”, or simply “system”) is formed of any suitable size, shape and design.

In the arrangement shown, as one example, vision impairment screening system 10 is configured to provide the state of the art with a vision screening system, vision impairment screening device, vision impairment screening system, processes, and methods of assessments, and methods of use.

In the arrangement shown, as one example, vision screening system 10 may comprise remote servers, databases, application servers, application databases, product databases, mobile applications, and/or computers; all of which in continuity or as separate acts fulfill the functions disclosed herein. Photo screening system 10 also includes, in the embodiment(s) depicted, a graphical user interface 12, a user (or patient), a second user (or examiner), a smart device 20, a camera 22, and a flash 24, an application server, and a mobile computing application, among other components, features, and functions.

In the arrangement shown, as one example, the main structure of system 10 also includes an attractor module 30 in one embodiment, an attractor module and a smart device case 130 in another embodiment, a sensor system 230, and communication and control components, among other components, features, and functionality.

### Graphical User Interface

In the arrangement shown, as one example, system 10 may include a graphical user interface 12. Graphical user interface 12 is formed of any suitable size shape and design and is configured to allow a user to view interact with, manipulate, and visually access environmental and system data and information, information related thereto, information related to a sensing environment including distance to a user, distance to a patient, lighting in the environment and more; and/or view various data for various surfaces and/or patients and/or add information to an vision screening system and/or environment and/or add information about an eye surface, retina or the like and/or object data and/or change the settings of the sensors and/or change the settings of the vision screening system operation.

In the arrangement shown, and in the attractor module embodiment, graphical user interface 12 is configured and included within the system and housing, and more specifically within the onboard computing system and/or computing system associated with the attractor module.

In another arrangement and embodiment, as also shown in a second example, the graphical user interface 12 is configured to operate through an application, or the like, on a smart device such as a mobile phone or smartphone, a tablet, a hand held device, a computer, or the like.

Graphical user interface 12 is an exemplary method by which systems of the present disclosure may determine vision and/or eye status, analyze for vision impairment, analyze for vision diseases and/or problems, both remotely and/or make programming changes to the operation of system 10. Employing graphical user interface 12, enhances a user's interactions with system 10 in the form of awareness and knowledge of data within the system 10. System 10 provides direct links to a user's and/or patient's history, settings, programming, and other information related to a particular patient and/or examiner and/or a particular medical or ophthalmological review.

In this way graphical user interface 12 provides for a means for a user to make adjustments to the operation and functionality of system 10. Some of these adjustments may include, but are not limited to, light settings, distance settings, camera settings, patient information, image quality adjustment, and more. Additionally, graphical user interface 12 provides direct interaction with the vision system, geometrical measurements, objects, system detection, system evaluation programs and automated analysis of an eye and/or retina, and more. Additionally, local geographic information with relation to the system can be added, viewed, or retrieved. Geographic information may include global positioning location and/or environmental data in the immediate room and/or environment.

Display of Graphical User Interface: In the arrangement shown, as one example, system 10 includes a graphical user interface 12. Graphical user interface 12 may include a display, which is configured to show and display information, including data, for review and interpretation by a user or plurality of users, or a plurality of users interacting with one another.

In the arrangement shown, as one example, an interactive user display is formed of a display screen, such as that of a desktop computer, laptop computer, monitor, tablet, smart phone, smart TV, projector, virtual reality display or any other device or form of a display. In the arrangement shown, as one example, interactive user display comprises a series of interactive user display pages, however, the interactive user display may consist of a single page or any other method of displaying information on a display as could be adapted to various size screens, devices, or user preferences. The interactive user display can display various patient and/or medical information which is retrieved and/or requested.

In the arrangement shown, as one example (and as further described herein), the attractor module may include a plurality of displays, including a first display, a second display, a third display, a fourth display, or more; as may aid in the analysis and/or vision screening processes (further described herein).

### User or Patient (or Plurality Thereof)

In the arrangement shown, as one example, system 10 includes a user. User may be any user interacting with or utilizing the system 10. This may include viewing, controlling, analyzing, manipulating, and/or interacting with system 10. Users are not limited to a single user but may be a plurality of users.

In the arrangement shown, a user 14 may also be referred to as a patient. In this way, the user 14 may be a user who is the object of the vision screening process. This user may have one eye or two eyes which are being examined as part of the vision screening process. The user may be self-analyzing and/or may also include a second or more users that are assisting with the image capture process and/or vision screening process.

### User or Examiner (or Plurality Thereof)

In the arrangement shown, as one example, system 10 includes a user. User (or examiner and/or vision care provider) may be any user interacting with or utilizing the system 10. A vision care provider may include, but is not limited to, an optometrist, a pediatrician, a physician, a primary care physician, a vision care professional, a nurse, and the like. This may include viewing, controlling, analyzing, manipulating, and/or interacting with system 10. Users are not limited to a single user but may be a plurality of users. In this circumstance a user or examiner may also include a remotely located individual or group of individuals who are examining the information gathered as part of the screening process. In this way, an examiner may include any medical professional, including but not limited to, a nurse, a physician, an opthamologist, an optometrist, other eye doctors, other medical doctors, or a programmer, a combination thereof, or the like.

### Smart Device

In the arrangement shown, as one example, system 10 may include a smart device (also referred to as a “mobile phone”, a “smartphone”, or simply a “device”). Smartphone 20 is formed of any suitable size, shape, and design and is configured to serve as an interface between the attractor and the user.

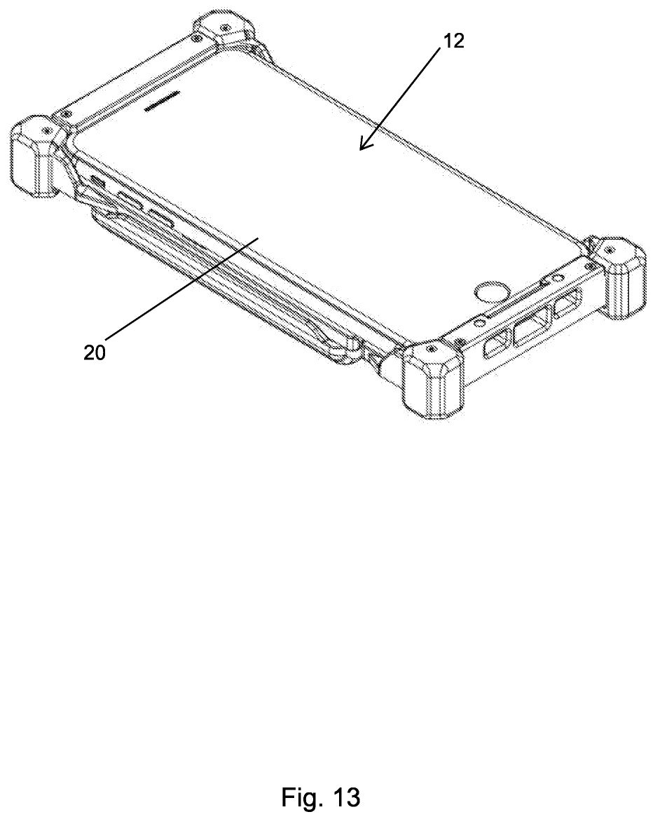

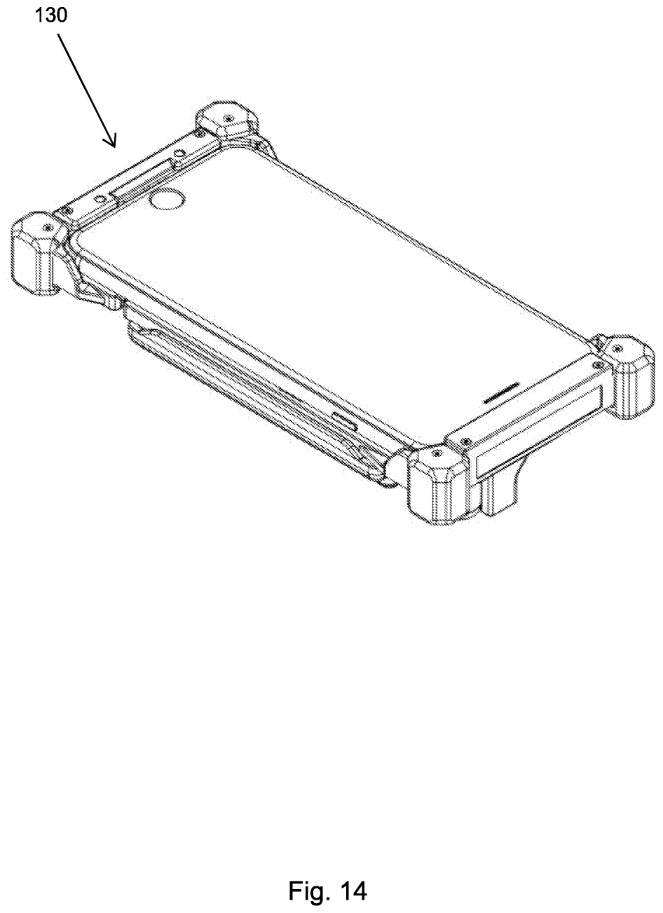

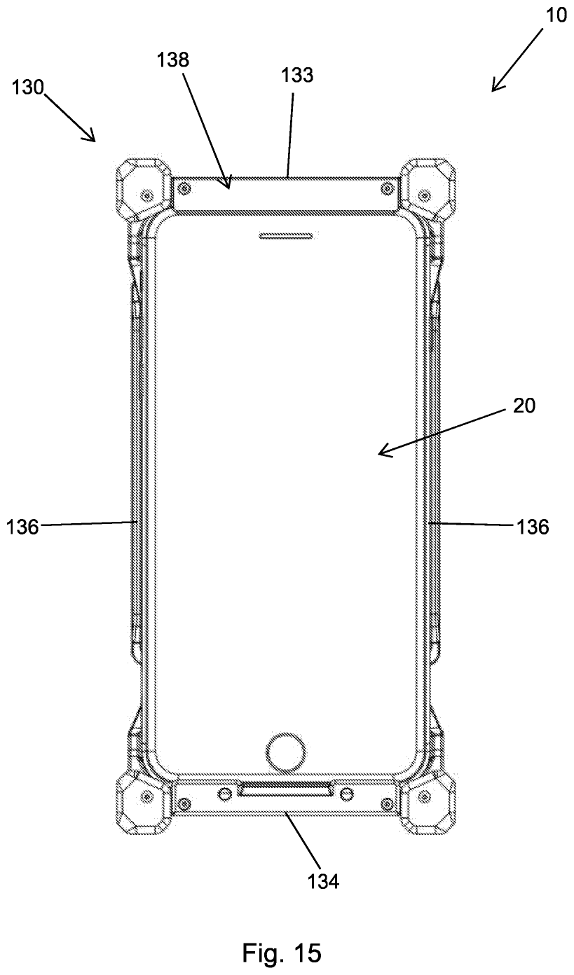

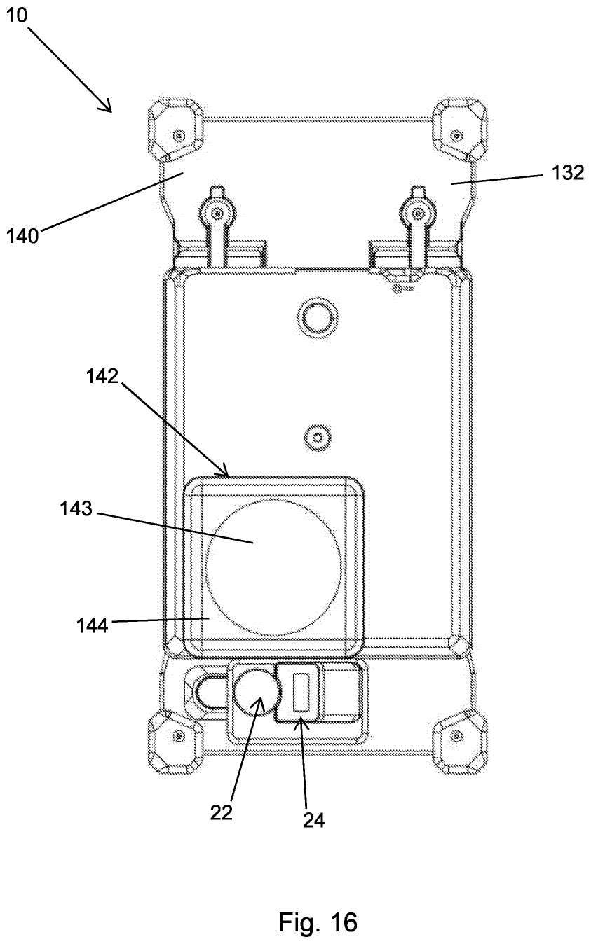

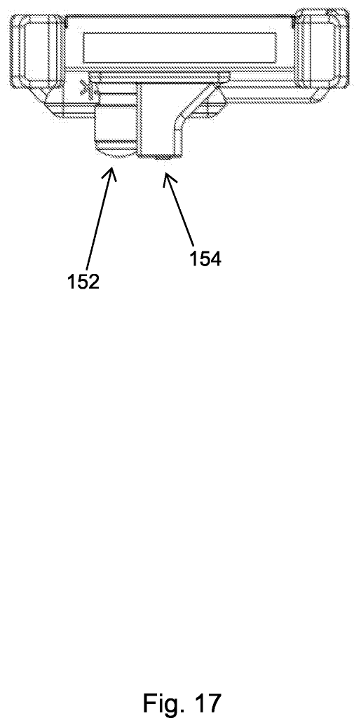

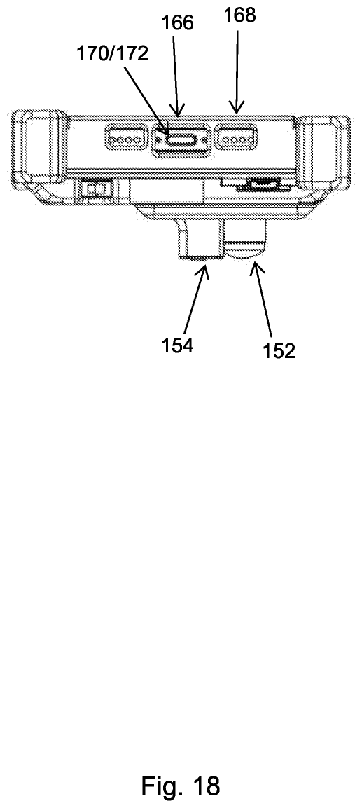

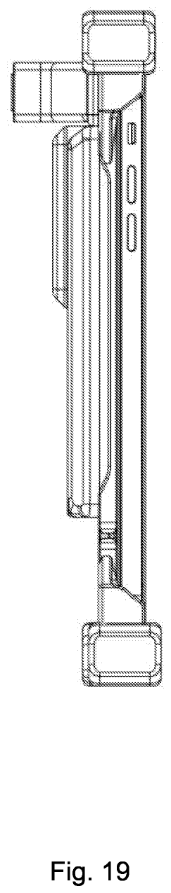

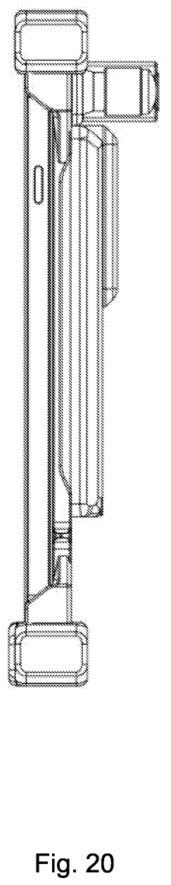

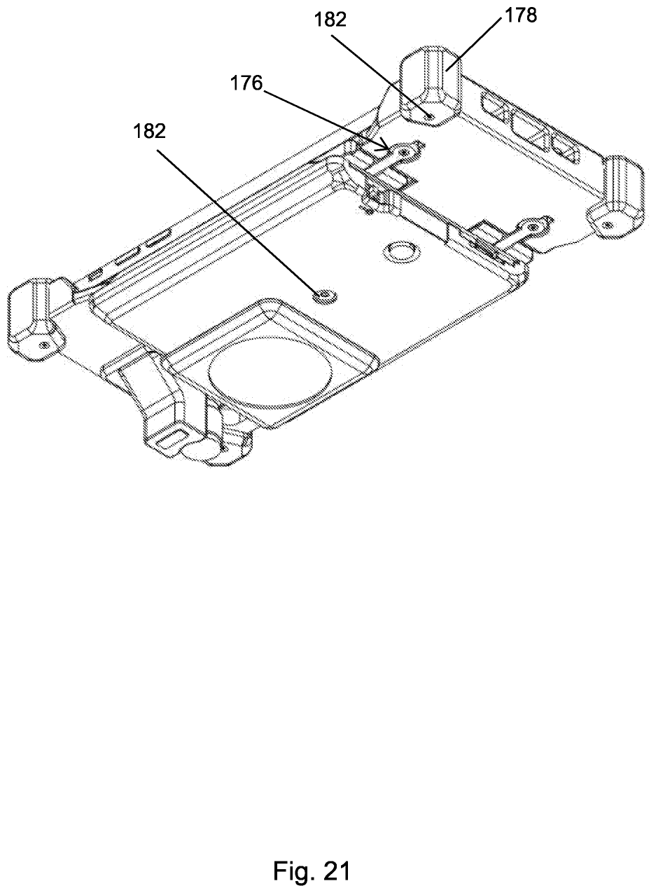

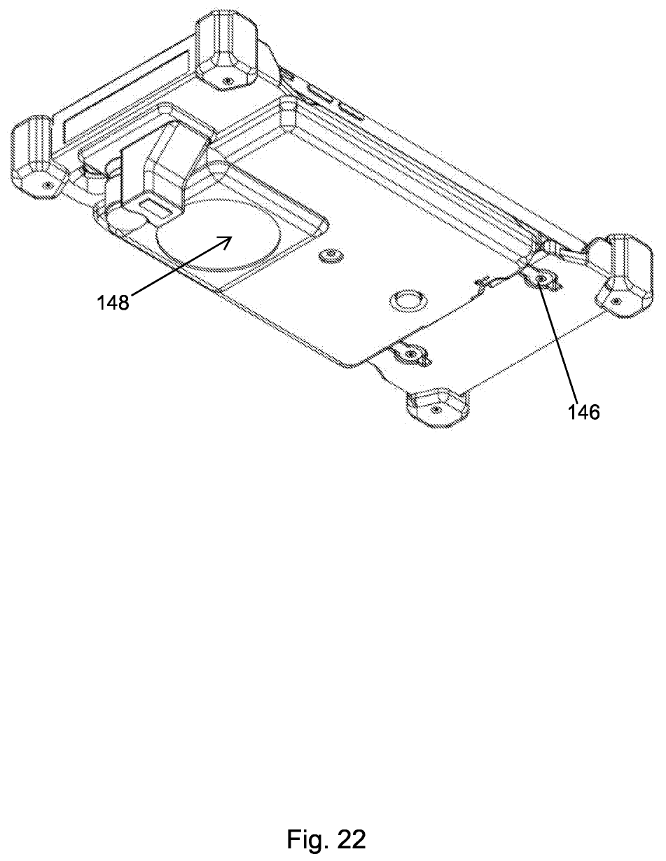

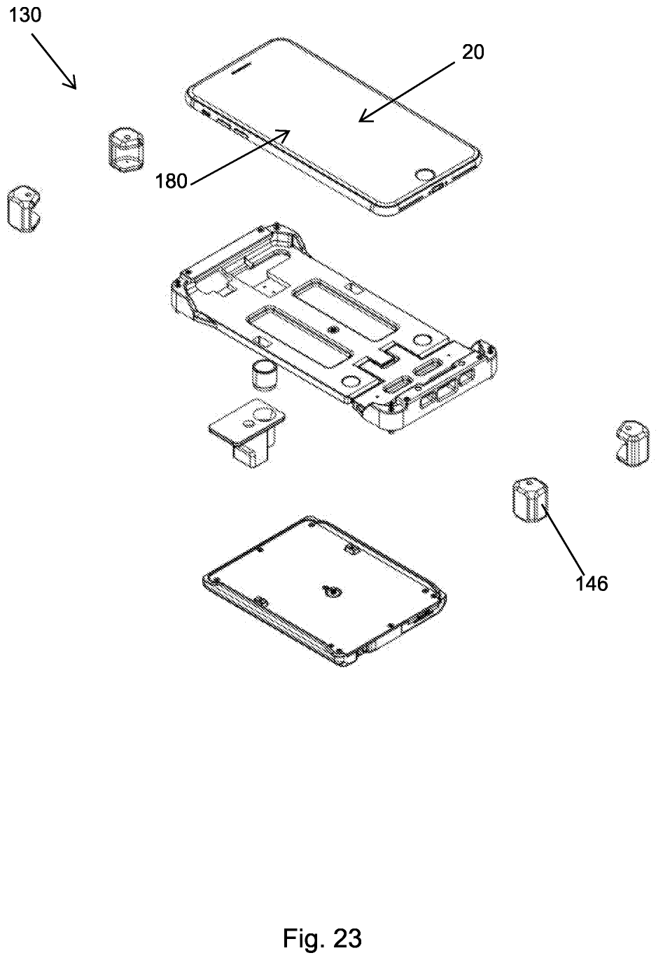

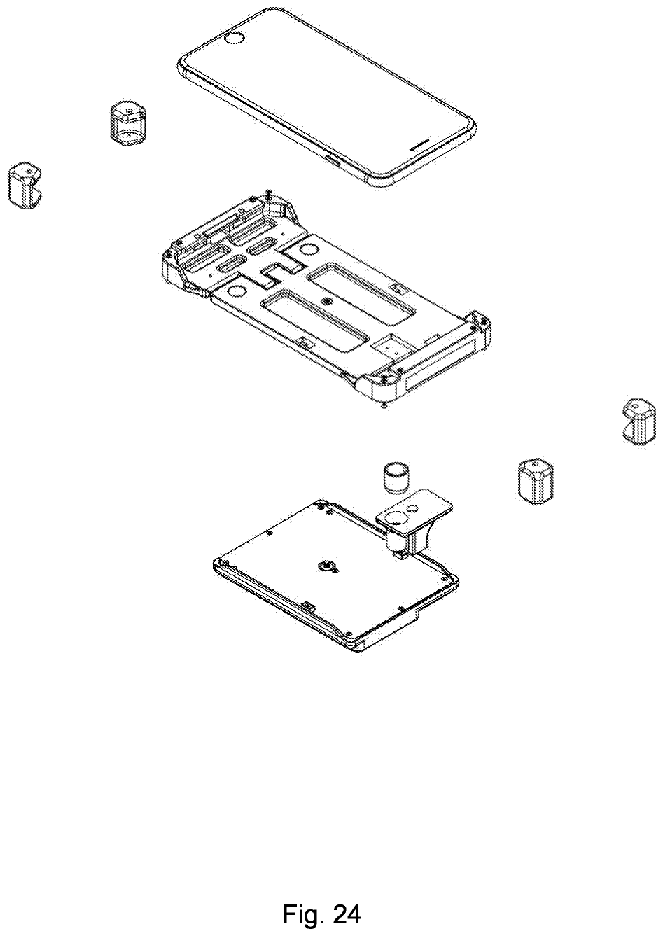

In this way, and as is shown in one embodiment (with specific reference to FIGS. 13 to 24), smart device 20 is included and configured to serve as a display screen and interaction feature. This may include, but is not limited to, programming graphical user interface 12, previewing images and/or data to be captured, modifying data which has been captured, viewing data which has been captured, entering data, manipulating the devices via zoom and other functionality, changing colors of attractor, modifying other settings of the hardware components of the attractor module (to be further described herein), and the like.

Furthermore, and in the arrangement shown, as one example, the smart device 20 is also configured to act as a potential power supply for the attractor module, and the like. In addition to acting as the power supply and the like, the smartphone 20 may also be configured to act as a camera and/or flash, among other functionality, and the like.

### Camera

In the arrangement shown, as one example, system 10 includes a camera 22 (also referred to as a “smartphone camera”, an “attractor module camera”, or simply a “camera feature”). Camera feature 22 is formed of any suitable size, shape, and design and is configured to capture images of a patient's eye and/or eyes to be used for vision screening.

In the arrangement shown, the camera may be included in the attractor module. Additionally, the camera 22 may include a lens specific to the photoscreening function to enhance performance. Additionally, in another embodiment, and as shown, the camera may be included as part of the smart device 20. In this case, and in another embodiment, the functionality of the camera of the smart device 20 may be modified by the system 10 for applicable uses in the vision impairment and/or vision screening process.

### Flash

In the arrangement shown, as one example, system 10 includes a flash 24 (also referred to as a “light feature”, a “flash feature”, or simply a “flash”). Light feature 24 is formed of any suitable size, shape, and design and is configured to provide lighting which will specifically light the eye and/or eyes of a patient for the capture images of a patient's eye and/or eyes to be used for vision screening.

In the arrangement shown, the flash 24 may be included in the attractor module. Additionally, the flash 24 may include a lens specific to the photoscreening function to enhance its performance. Additionally, in another embodiment, and as shown, the flash 24 may be included as part of the smart device 20. In this case, and in another embodiment, the functionality of the flash 24 of the smart device 20 may be modified by the system 10 for applicable uses in the vision impairment and/or vision screening process.

### Attractor Module

In the arrangement shown, as one example, system 10 includes an attractor module 30 (also referred to as a “attractor system”, a “vision screening device”, or simply an “attractor”). Attractor system 30 is formed of any suitable size, shape, and design and is configured to attract the gaze of a user, capture images for analysis, provide lighting for image capture, and transmit and/or save data for a user's interaction, as related to the images captured and the like.

Furthermore, the attractor module and associated computer functionality may also include and be configured to provide an automated analysis with machine learning capabilities based on a predetermined set of rules which are applicable to vision screening. This analysis may include, but is not limited to, patterned analysis and recognition based on common ailments and/or common appearances within eye and/or vision analysis. As strictly an example, and for ease of explanation, the automated analysis may include a check for amblyopia, which can be determined mathematically in many cases.

Furthermore, and in the arrangement shown, as one example, the attractor module 30 is configured to provide all the needed functionality in a single housing such that the system can operate without the need for a smart device or other components.

Additionally, and in the arrangement shown, as one example, attractor system 30 is configured to act as a complete screening device capable of screening and providing analysis, and delivering results and/or conveying results to a medical professional for further screening. Furthermore, the attractor module 30 is configured to provide analysis of a medical professional after the analysis is provided to that medical professional.

Said another way, and in the arrangement shown, as one example, the attractor module 30 is configured to photograph, detect risk factors, and perform other photoscreening functionality, including analysis, and detection of vision impairment and/or vision disease. The attractor module 30 is configured to use flash photography and/or photography and/or image capture to capture images properly (as needed for vision screening) and detect other key features of an image, including but not limited to, dimensional aspect ratios and establish risk criteria and the like.

In the arrangement shown, as one example, the attractor module 30 is configured to attract the gaze and/or attention of a patient such that the gaze is optimized for vision screening. This includes, but is not limited to attracting the gaze of a user toward the camera and focused at infinity. Furthermore, this includes the direction a patient is looking and/or focusing (which is critical for the capture of an image for vision screening). Furthermore, this includes measurements made when the subjects and/or patients gaze deviates away from the camera. Furthermore, this includes illuminating the patient and the eye properly and at the proper distance.

Furthermore, the attractor module 30 includes the ability to operate in the red region of the light spectrum, which allows for image capture and/or reflection capture of the enlarged pupil in a darkened room. In this way, the red light does not cause pupil constriction during attraction of the patient's gaze and enhances the image collection process. Pupil dilation and/or pupil constriction levels are important in capturing the proper images in photoscreening. Said another way, the red spectrum feature and/or infra-red part of the spectrum and/or red spectrum (or “red light feature”) does not cause pupil constriction and/or causes a scene to be lit without causing pupil constriction, and has no effect on flash.

Similarly, and in this way, the attractor module 30 includes a flash system which is optimized for image gathering in the vision screening process. In this way, the flash module may utilize light emitting diodes of various spectrums or xenon flash, for example. Furthermore, the flash module can be configured to be triggered from the optical sensor (further described herein) and/or the on board camera flash and/or through bluetooth and/or other transmission and/or timing and/or communication. Alternatively, the flash module can be configured to utilize a lens for concentrating illumination and/or an optical wavelength filter for its spectrum. Said another way, the flash module (further described herein) is optimized for the vision screening process.

In the arrangement shown, the standalone attractor module 30 includes a housing 32 which extends a length from a top 33 to a bottom 34, between opposing sides 36. In the arrangement shown, the attractor module 30 extends a length from a first end (or a “front end”) to a second end (or a “rear end”, or a “back end”). Further, having an on/off switch 74.

Diffuser: In the arrangement shown, as one example, the attractor module 30 also includes a diffuser 42. Diffuser 42 is formed of any suitable size, shape, and design and is configured to optimize the gaze of a user through changing the light and/or light display in a way to focus the gaze of a user in a certain direction and at a particular distance. In this way, the diffuser 42 is configured to diffuse light in a particular way. Diffuser 42 may also include a diffuser liner 43 and a diffuser housing 44, among a plurality of attachment features 46.

In the arrangement shown, as one example, a single diffuser is utilized. However, a plurality of diffusers are hereby contemplated for use. In this way, various angles of a user's vision may be desired to be captured such that a plurality of diffusers located across the front end of the housing are hereby contemplated for use, including but not limited to, adjacent locations of diffusers. This may include a single diffuser, two diffusers, three diffusers, four diffusers, five diffusers, six diffusers, seven diffusers, eight diffusers, or more and/or different types of diffusers. Furthermore, interchangeable diffusers are hereby contemplated for use. Furthermore, different diffusers utilized may have different properties such as in light modification, more or less diffusivity, and the like.

In the arrangement shown, as one example, the attractor module 30 also includes a plurality of lights. The plurality of lights 48 may be part of the diffuser or may be separately located in order to attract attention and/or light the eye and/or eyes of a user. In this way, a plurality of lights are hereby considered as part of the attractor module 30 for a variety of purposes (including but not limited to serving flash purposes).

Power Supply: In the arrangement shown, attractor module 30 also includes a power supply. Power supply is formed of any suitable size, shape and design and is configured to provide power to the various components of the attractor module 30. Power supply in the arrangement shown, does not move such that other mechanical components of the system may “move”. Instead, the power supply is configured as a source of energy for the attractor module 30.

In the arrangement shown, as one example, power supply is formed of a plurality of rechargeable batteries. The rechargeable battery, or storage battery, is an electrical battery designed to be charged, discharged, and recharged as many times as needed, as opposed to a disposable or primary batter, which is disposed of after use. However, a primary battery or disposable battery is also hereby contemplated for use. The rechargeable battery, in the arrangement shown, is formed of one or more electrochemical cells. In the arrangement shown, as one example, power supply is formed of a rechargeable battery. However, the power supply may also consist of a plurality of rechargeable batteries and/or a plurality of batteries.

In the arrangement shown, as one example, the power supply may also be formed of other power types and/or generators including, but not limited to solar power supply, or other elements into mechanical energy, as well as other power supply types.

Furthermore, in the arrangement shown, power supply may also include all necessary components, whether wires, conductors, cases, holders, and the like which provide for necessary connections and stabilization for power supply components.

Camera Module: In the arrangement shown, as one example, attractor module 30 includes a camera module. Camera module is formed of any suitable size, shape, and design and is configured to capture images of the eye and/or eyes of a patient for the purpose of vision screening and analysis. Camera module may include various sensors, camera elements, lenses, focusing apparatuses, shutters, and the like which are configured to capture images with optimized resolution and image quality.

Furthermore, in the arrangement shown as one example, a single camera module is utilized and configured. However, a plurality of camera modules are hereby contemplated for use, including but not limited to, a single camera module, two camera modules, three camera modules, four camera modules, five camera modules, or more and/or different types of cameras.

Furthermore, in the arrangement shown, as one example, the camera module is included in the attractor module 30. However, the camera module may be a standalone feature which is operably connectable to the attractor module 30.

Flash Module: In the arrangement shown, as one example, the attractor module 30 also includes a flash module. Flash module is formed of any suitable size, shape, and design and is configured to provide the flash and/or flashes necessary for optimal image capture. Furthermore, the flash module and/or a secondary flash module may also provide lighting and/or instant lighting needed to attract the optimal gaze of a patient.

In this way, the flash module may utilize a plurality of light emitting diodes or xenon flashes. Furthermore, the flash module can be configured to be triggered from the optical sensor (further described herein) and/or the on board camera flash and/or through bluetooth and/or other transmission and/or timing and/or communication. In this way, the flash module is configured to utilize a lens and/or an optical wavelength. Said another way, the flash module is optimized for the vision screening process.

In the arrangement shown, as one example, the attractor module 30 includes a single flash module. However, any other number of flash modules as may aid in image capture and/or patient gaze focus attraction are hereby contemplated for use, including but not limited to, a single flash module, two flash modules, three flash modules, four flash modules, five flash modules, six flash modules, seven flash modules, or more flash modules and/or different types of flash modules. Furthermore, the flash modules may be oriented to produce the red reflex. For this reason, various orientations of a flash module and/or flash modules on various axes, in various directions, and the like are hereby contemplated for use. Furthermore, flash modules may be utilized to generate different light spectrums for improved sensitivity, environmental factors, and improved analysis. Furthermore, different flash modules, which may also be interchangeable, may serve different purposes in analysis and collection of data, such as providing different durations of flash, different timing of each flash for various uses. In this way, various images can be captured and/or multi-image capture operations can be performed.

Furthermore, in the arrangement shown, as one example, the flash module is included in the attractor module 30. However, the flash module may be a standalone feature which is operably connectable to the attractor module 30.

In the arrangement shown, as one example, the attractor module 30 may also include an onboard computing system (to be further described herein), a transceiver (to be further described herein), a communications system and/or communications module (to be further described herein, along with a first display, a second display, or more displays (further described herein), and a sensor system (further described herein), among other components, features, and functionality.

### Alternative Embodiment

### Case Attractor Module

In an alternative embodiment and in another arrangement shown, as another example, and with reference to FIGS. 13 to 24, system 10 includes a cased attractor module 130 (also referred to as a “case attractor system”, a “adapter vision screening device”, “retrofit vision screening system”, “adaptive attractor”, or simply a “case attractor”). Case attractor system 130 is formed of any suitable size, shape, and design and is configured to attract the gaze of a user, capture images for analysis, provide lighting for image capture, and may transmit and/or save data for a user's interaction—transmitting this data through an attached smart device or the like—as related to the images captured, data captured and analyzed, and the like.

Furthermore, the adaptive attractor module 130 and associated computer functionality may also include and be configured to provide an automated analysis with machine learning capabilities based on a predetermined set of rules which are applicable to vision screening—transmitting this data through an attached smart device or the like. This analysis may include, but is not limited to, pattern analysis and recognition based on reference data, common ailments and/or common appearances within eye and/or vision analysis. As strictly an example, and for ease of explanation, the automated analysis may include a check for amblyopia, for which risk can be determined mathematically with photoscreening.

Furthermore, and in the arrangement shown, as one example, the cased attractor module 130 is configured to provide all the needed functionality in an adaptive system that can be attached or connected to a smart device, such as a smartphone, and the like. In this way, and in combination with a smart device, the case attractor system 130 is configured to act as a complete screening device capable of screening and providing analysis, delivering results and/or conveying results to a medical professional for further screening. Furthermore, the case attractor module 130 is configured to provide analysis of a medical professional after the analysis is provided to that medical professional.

Said another way, and in the arrangement shown, as one example, the adaptor vision screening device 130 (or “attractor screening vision device”) is configured to photograph, detect risk factors, and perform other photoscreening functionality, including analysis, and detection of vision impairment and/or vision disease. The case attractor module 130 is configured to use flash photography and/or photography and/or image capture to capture images properly (as needed for vision screening) and detect other key features of an image, including but not limited to, dimensional aspect ratios and establish risk criteria and the like.

In the arrangement shown, as one example, the case attractor module 130 is configured to attract the gaze and/or attention of a patient such that the gaze is optimized for vision screening. This includes, but is not limited to infinite depth and/or infinite gaze of a user. Furthermore, this includes the direction a patient is looking and/or focusing (which is critical for the capture of an image for vision screening). Furthermore, this includes measurements made when the subjects and/or patients gaze deviates away from the camera. Furthermore, this includes illuminating the patient and the eye properly and at the proper distance.

Furthermore, and alternatively, the case attractor module 130 includes a red light spectrum feature which allows for image capture and/or reflection capture of the enlarged pupil in a darkened room. In this way, the red light does not cause pupil constriction even during the flash and/or image collection process. Pupil dilation and/or pupil constriction levels are important in capturing the proper images reflected from the retina for vision screening.

Similarly, and in this way, the case attractor module 130 includes a flash system which is optimized for image gathering in the vision screening process. In this way, the flash module may utilize light emitting diodes or xenon flash. Furthermore, the flash module can be configured to be triggered from the optical sensor (further described herein) and/or the on board camera flash and/or through bluetooth and/or other transmission and/or timing and/or communication. In this way, the flash module is configured to utilize a lens and/or an optical wavelength. Said another way, the flash module (further described herein) is optimized for the vision screening process.

In the arrangement shown, the retrofit attractor module 130 includes a housing 132 which extends a length from a top 133 to a bottom 134, between opposing sides 136. In the arrangement shown, the cased attractor module 130 extends a length from a first end 138 (or a “front end”) to a second end 140 (or a “rear end”, or a “back end”).

Diffuser: In the arrangement shown, as one example, the retrofit attractor module 130 also includes a diffuser 142. Diffuser 142 is formed of any suitable size, shape, and design and is configured to optimize the gaze of a user through changing the light and/or light display in a way to focus the gaze of a user in a certain direction and at a particular distance. In this way, the diffuser 142 is configured to diffuse light in a particular way. Diffuser 142 may also include a diffuser liner 143 and a diffuser housing 144, among a plurality of attachment features 146.

In the arrangement shown, as one example, a single diffuser is utilized. However, a plurality of diffusers are hereby contemplated for use. In this way, various angles of a user's vision may be desired to be captured such that a plurality of diffusers located across the front end of the housing are hereby contemplated for use, including but not limited to, adjacent locations of diffusers. This may include a single diffuser, two diffusers, three diffusers, four diffusers, five diffusers, six diffusers, seven diffusers, eight diffusers, or more and/or different types of diffusers.

In the arrangement shown, as one example, the retrofit attractor module 130 also includes a plurality of lights. The plurality of lights may be part of the diffuser or may be separately located in order to attract attention and/or light the eye and/or eyes of a user. In this way, a plurality of lights are hereby considered as part of the cased attractor module 130 for a variety of purposes (including but not limited to serving flash purposes). Furthermore, in an alternative embodiment, the diffuser 142 may be configured to utilize the flash of the smart device and/or manipulate the flash of the smart device.

Power Supply: In the arrangement shown, retrofit attractor module 130 may also include a power supply. However, the retrofit attractor module 130 is designed to rely on the power supply of the smart device. In this way, the power supply may be a power cord attached to a smart device for power transmittance and/or other power transmission such as wireless charging and/or wireless power transfer. In this way, power supply is formed of any suitable size, shape and design and is configured to provide power to the various components of the case attractor module 130.

Alternatively, power supply may also be an onboard power supply. In this alternative arrangement, as one example, power supply may be formed of a plurality of rechargeable batteries. The rechargeable battery, or storage battery, is an electrical battery designed to be charged, discharged, and recharged as many times as needed, as opposed to a disposable or primary batter, which is disposed of after use. However, a primary battery or disposable battery is also hereby contemplated for use. The rechargeable battery, in the arrangement shown, is formed of one or more electrochemical cells. In the arrangement shown, as one example, power supply is formed of a rechargeable battery. However, this alternative power supply may also consist of a plurality of rechargeable batteries and/or a plurality of batteries.

In this alternative arrangement, as one example, the power supply may also be formed of other power types and/or generators including, but not limited to solar power supply, or other elements into mechanical energy, as well as other power supply types.

Furthermore, in the arrangement shown, power supply may also include all necessary components, whether wires, conductors, cases, holders, and the like which provide for necessary connections and stabilization for power supply components.

Camera Module: In the arrangement shown, as one example, case attractor module 130 includes a camera module 152. Camera module 152 is formed of any suitable size, shape, and design and is configured to capture images of the eye and/or eyes of a patient for the purpose of vision screening and analysis. Camera module 152 may include various sensors, lenses, focusing apparatuses, shutters, and the like which are configured to capture images.

Furthermore, in the arrangement shown as one example, a single camera module 152 is utilized and configured. However, a plurality of camera modules are hereby contemplated for use, including but not limited to, a single camera module, two camera modules, three camera modules, four camera modules, five camera modules, or more and/or different types of camera modules.

Furthermore, in the arrangement shown, as one example, the camera module 152 is included in the case attractor module 130. However, the camera module 152 may be a standalone feature which is operably connectable to the case attractor module 130.

Flash Module: In the arrangement shown, as one example, the retrofit attractor module 130 also includes a flash module 154. Flash module 154 is formed of any suitable size, shape, and design and is configured to provide the flash and/or flashes necessary for optimal image capture. Furthermore, the flash module and/or a secondary flash module may also provide lighting and/or instant lighting needed to attract the optimal gaze of a patient.

In this way, the flash module 154 may utilize a plurality of light emitting diodes or xenon flashes. Furthermore, the flash module 154 can be configured to be triggered from the optical sensor (further described herein) and/or the on board camera flash and/or through bluetooth and/or other transmission and/or timing and/or communication. In this way, the flash module 154 is configured to utilize a lens and/or an optical spectrum. Said another way, the flash module is optimized for the vision screening process.

In the arrangement shown, as one example, the retrofit attractor module 130 includes a single flash module 154. However, any other number of flash modules as may aid in image capture and/or patient gaze focus attraction are hereby contemplated for use, including but not limited to, a single flash module, two flash modules, three flash modules, four flash modules, five flash modules, six flash modules, seven flash modules, or more flash modules and/or different types of flash modules.

Furthermore, in the arrangement shown, as one example, the flash module is included in the case attractor module 130. However, the flash module 154 may be a standalone feature which is operably connectable to the retrofit attractor module 130.

In the arrangement shown, as one example, the retrofit attractor module 130 utilizes power and functionality of the smart device. Some of these smart device systems which are utilized may include the smart device computational power, the smart device transceiver, the smart device communications system, the smart device display and the smart device sensor system.

In an alternative to the embodiment, the retrofit attractor module 130 may not have a full camera module, but instead utilize a camera filter, or additional lenses, or the like to utilize the camera functionality of the smart device.

Similarly, in an alternative to the embodiment, the retrofit attractor module 130 may not have a full flash module, but instead utilize a flash filter, or additional diffusers and the like to utilize the flash functionality of the smart device. Furthermore, the flash could include a lens. Furthermore, the flash might include a plurality of lenses and/or filters.

In an alternative embodiment, as another example, the retrofit attractor module 130 may also include these components as a standalone from the smart device. These components may include, but are not limited to, an onboard computing system (to be further described herein), a transceiver (to be further described herein), a communications system and/or communications module (to be further described herein, along with a first display, a second display, or more displays (further described herein), and a sensor system (further described herein), among other componentes, features, and functionality.

In the arrangement shown, as one example, the retrofit attractor module 130 also includes a plurality of charging apertures 166, a plurality of sound apertures 168, a power connection feature 170, a support connection 172, an on/off switch 174, a plurality of adjustment features 176, a plurality of attachment features 178, a mounting feature 180, and a sensor system, among other features, components, and functionality.

### Sensor System

In the arrangement shown, as one example, system 10 includes a sensor system. Sensor system is formed of any suitable size, shape, and design and may include one or more sensors and/or one or more sensing technologies. In the arrangement shown, as one example, the sensor system is configured to detect and communicate information related to system 10 as well as the surroundings and/or environment of system 10.

In the arrangement shown, as one example, various sensors are utilized within system 10 to detect system needs such as brightness levels of an environment, distance of a subject, measurements of a pupil, and other operating and/or vision screening parameters within for the effective operation of system 10. Furthermore, and said another way, various sensors may be utilized for detecting ambient room light for optimization in the photoscreening process.

In the arrangement shown, as one example, a sensory system and perhaps a LIDAR sensor system may be utilized to understand the environment surrounding system 10 and/or the environment and/or subject and/or patient system 10 is engaging with. This may include both distance and imaging sensors and/or camera sensors.

### Other Sensors

In addition to the distance and image sensors discussed herein, system 10 may also include other sensors, such as temperature sensors, moisture sensors, heat sensors, light sensors, motion sensors, and other sensors. In the arrangement shown, as one example, system 10 includes at least one other sensor. Other sensors are formed of any suitable size, shape and design and are configured to facilitate sensing of the subject and/or patient and/or environment and converting the characteristics of the outside space and/or environment into computer readable information. Other sensors are used to detect and respond to some type of input from the physical environment and/or the patient information.

Other sensors may be used for sensing a single component of a subject and/or patient and/or environment, or alternatively another sensor may be used to sense a plurality of components of a subject and/or patient and/or environment. For example, the specific input of other sensors may be light, heat, motion, moisture, pressure, or any one of a great number of other surface and/or environmental information.

Another sensor is a device, module, or subsystem whose purpose is to detect events or changes in its environment and/or patient and/or patient over time and send the information to other electronics, frequently a computer processor. The output of another sensor is generally a signal that is generally converted to human-readable display at the sensor location or transmitted electronically over a network for reading or further processing. Both analog sensors and/or digital sensors are hereby contemplated for use. In one arrangement, another sensor and/or microsensor sends information to a processor for use with other electronics.

### Computing Platform

In the arrangement shown, as one example, system 10 includes a computing platform (or “computer”, or “computer platform”). Computing platform is formed of any suitable size, shape, and design and is configured to provide computing support, power, and computing processing for both onboard computing functionality as well as communication for off-board or server computing functionality. In this way, an onboard computing system, among other components and features.

In the arrangement shown, as one example, system 10 includes a computer. Computers are formed of any suitable size, shape, and design and are configured to provide for the main off-board computing processing and implementation of computer handling of data from data gathering performed within the environment. The data processing may include, but is not limited to, artificial intelligence of sorting and organizing images gathered which may include stitching and object recognition and/or layer and layer recognition of continuous scans of a surface and/or patient and/or eye and/or eyeballs and surrounding areas of the eye and/or retinas and other components of system 10.

### Onboard Computing System

In one arrangement, as is shown, system 10 includes an onboard computing system (or “onboard computing device”). Onboarding computing system is formed of any suitable size, shape, and design and configured to handle onboard computing operations, as are necessary for the operation of the attractor module 30 and/or retrofit attractor module 130 and/or system 10. Onboarding computing device is connected with electronic network and/or database and/or server or cloud via communication means, bluetooth communication, bluetooth low energy chip (BLE onboard), and may include a processor, a memory, a microcontroller, a printed circuit board, a microprocessor, a receiver/transceiver, may include at least one antenna, and a global positioning system, among other components.

Computing device may be formed of and/or include any computing device capable of displaying and manipulating data in the manners described herein. Computing device may include for example a desktop computer, a laptop computer, a tablet, smart phone, or any other computing device or other interactive device.

Computing device may be a single consolidated component, or alternatively, computing device may be formed of a plurality of interconnected components that may be co-located or located at different geographic locations. Computing device may be cloud based or it may be hardware based, or cloud capable. In addition, the connected components of computing device, including processor, memory, software and interactive user display, may be co-located with computing device or located at different geographic locations, independent control features of the system, and the like. That is, computing device may be made of any form of a device or system that individually or collectively performs the computing operations of system 10.

Printed Circuit Board: In the arrangement shown, as one example, system 10 may include a printed circuit board (“PCB”). PCB is formed of any suitable size, shape and design and is configured to facilitate carrying and/or holding other components and/or parts necessary to carry out various computation and/or related functions of system 10. PCB, as one example, might be a surface mounted PCB or a through-hole PCB. PCB, as one example, is green and facilitates connecting the components and/or parts of system 10 by the use of traces and or vias. Traces are formed of any suitable size, shape and design and are configured as lines electrically connecting the components and/or parts of system 10. Vias are formed of any suitable size, shape and design and are configured as holes that connect layers of traces together. Generally, as in shown, traces and vias are soldered to connect the components and/or parts to the PCB.

In an alternative embodiment, system 10 may not include an onboard computer device 240, but instead houses only a receiver and/or transceiver for sending and/or receiving information which is both sensed and/or information which is sensed and/or captured by the image capturing devices of system 10. In this way, system 10 may be connected to a mobile computing device via a cellular connection, a direct connection, or other connections which provide a means of receiving and/or sending communications.

Microprocessor: Microprocessor is any computing device that receives and processes information and outputs commands according to instructions stored in memory. Memory is any form of information storage such as flash memory, RAM memory, a hard drive, or any other form of memory. Memory may be included as a part of or operably connected to a microprocessor. A receiver/transceiver is connected to a microprocessor. A receiver is used if one way communication is utilized, whereas a transceiver is used if two-way communication is utilized (hereinafter “transceiver”). Receiver/transceiver is connected with an antenna, such as a monopole antenna, a loop antenna, a fractal antenna, or any other form of an antenna. Antenna receives wireless signals from any other device, transmits these signals to receiver/transceiver which processes these signals and then transmits these processed signals to microprocessor which processes these signals according to instructions stored in memory. In one arrangement, system 10 re-transmits operating commands signals through receiver/transceiver so as to similarly control over-the-air communication. Communication is any form of wireless signals, or wired signals, through the air, such as a conventional remote signal, a cell phone, a wireless device, an internet connected device, a hard-wired device, or any other device capable of transmitting remote control signals.

Memory: In the arrangement shown, as one example, system 10 includes a memory. Memory may be formed of any suitable size, shape and design and is configured to facilitate selective storage and retrieval of data (including data) in association with computing devices, processors, software and interactive user display. Memory may be a single component, such as a single chip or drive or other memory device, or alternatively memory may be formed of a plurality of memory or storage components that are connected to one another that may be co-located or located at different geographic locations.

### Remote Computing System

In one arrangement, as is shown, system 10 includes a remote computing system (or “remote computing device”). Remote computing device is formed of any suitable size, shape, and design and configured to handle offboard computing operations, as are necessary for the operation of the attractor module 30 and/or cased attractor module 130 and the operations of system 10. Remote computing device is connected with electronic network and/or database and/or server or cloud via communication means and includes a processor, a memory, a microcontroller, a printed circuit board, a microprocessor, a receiver/transceiver, may include at least one antenna, a power supply, and a communications system, among other components.

Computing devices may be formed of any computing device capable of displaying and manipulating data in the manners described herein. Computing devices may include for example a desktop computer, a laptop computer, a tablet, smart phone, or any other computing device or other interactive device.

Computing devices may be a single consolidated component, or alternatively, computing devices may be formed of a plurality of interconnected components that may be co-located or located at different geographic locations. Computing devices may be cloud based or it may be hardware based, or cloud capable. In addition, the connected components of computing devices, including processor, memory, software and interactive user display, may be co-located with computing devices or located at different geographic locations. That is, computing devices may be made of any form of a device or system that individually or collectively performs the computing operations of system 10.

Printed Circuit Board: In the arrangement shown, as one example, system 10 includes a printed circuit board (“PCB”). PCB is formed of any suitable size, shape and design and is configured to facilitate carrying and/or holding other components and/or parts necessary to carry out various computation and/or related functions of system 10. PCB, as one example, might be a surface mounted PCB or a through-hole PCB. PCB, as one example, is green and facilitates connecting the components and/or parts of system 10 by the use of traces and or vias. Traces are formed of any suitable size, shape and design and are configured as lines electrically connecting the components and/or parts of system 10. Vias are formed of any suitable size, shape and design and are configured as holes that connect layers of traces together. Generally, as in shown, traces and vias are soldered to connect the components and/or parts to the PCB.

In an alternative embodiment, system 10 may not include an onboard computer device 180, but instead houses only a receiver and/or transceiver for sending and/or receiving information which is both sensed and/or information which is sensed and/or captured by the image capturing devices of system 10. In this way, system 10 may be connected to a mobile computing device via a cellular connection, a direct connection, or other connections which provide a means of receiving and/or sending communications.

Microprocessor: Microprocessor is any computing device that receives and processes information and outputs commands according to instructions stored in memory. Memory is any form of information storage such as flash memory, RAM memory, a hard drive, or any other form of memory. Memory may be included as a part of or operably connected to a microprocessor. A receiver/transceiver is connected to a microprocessor. A receiver is used if one way communication is utilized, whereas a transceiver is used if two-way communication is utilized (hereinafter “transceiver”). Receiver/transceiver is connected with an antenna, such as a monopole antenna, a loop antenna, a fractal antenna, or any other form of an antenna. Antenna receives wireless signals from any other device, transmits these signals to receiver/transceiver which processes these signals and then transmits these processed signals to microprocessor which processes these signals according to instructions stored in memory. In one arrangement, system 10 re-transmits operating commands signals through receiver/transceiver so as to similarly control over-the-air communication. Communication is any form of wireless signals, or wired signals, through the air, such as a conventional remote signal, a cell phone, a wireless device, an internet connected device, a hard-wired device, or any other device capable of transmitting remote control signals.

Memory: In the arrangement shown, as one example, system 10 includes a memory. Memory may be formed of any suitable size, shape and design and is configured to facilitate selective storage and retrieval of data (including data) in association with computing devices, processors, software and interactive user display. Memory may be a single component, such as a single chip or drive or other memory device, or alternatively memory may be formed of a plurality of memory or storage components that are connected to one another that may be co-located or located at different geographic locations.

### Application Server

In the arrangement shown, as one example, system 10 comprises remote servers, databases, and/or computers that fulfill the functions disclosed and described herein. In the embodiment depicted, system 10 may also comprise an application server, and particularly with respect to the cased attractor module 130. Application server may comprise one or more computer systems adapted to transmit and receive data regarding selected datasets related to various users and/or datasets related to multiple users. Application server is adapted to query databases with unique identification codes to retrieve image information and associated surface information related to various surfaces system 10 has moved over and/or through and gathered data in, and/or has monitored and/or is monitoring.

Application servers may transmit environmental data and/or surface related documents with respect to a single environment and/or multiple environments. Application server is also adapted to query a database. This query includes receiving and sending surface identification and environmental data. Additionally, application servers may communicate with a cloud computing system or a mobile application, which can also be adapted to present the data in a form conducive to being viewed on a mobile device and/or handheld device.

As one of ordinary skills in the art may understand, application server, database, and other databases mentioned herein may be implemented in one or more servers. Furthermore, each may be on multiple servers to increase system efficiency, especially when handling large data gathering, following extended guidelines, extended ranges, ranges discussed herein. Additionally, multiple servers may have mirrored data to prevent data impairment in case of disk failure and/or to decrease access and response times for database queries. In alternative embodiments, application server 600, and other database procedures may be carried out on computer-readable instructions and data stored on the customer's mobile computing device.

In addition to the above identified features, options, controls, and components, system 10 may also include other features and functionalities, among other options, controls, and components.

### In Operation

In one example, a user will attract the needed gaze of a patient with the attractor module. The user will then capture images of the eye of a user for the vision screening process. The images will be analyzed for vision impairment and/or vision disease and/or other concerns before providing a diagnosis and/or recommendations. These operations and others which are apparent from the disclosure herein.

It will be appreciated by those skilled in the art that other various modifications could be made to the system, process, and method of use without parting from the spirit and scope of this disclosure. All such modifications and changes fall within the scope of the claims and are intended to be covered thereby.

What is claimed:

1. A vision screening system, comprising:
an attractor module;
the attractor module having a graphical user interface;
the attractor module extending a length from a first end to a second end;
the attractor module having a housing;
wherein the graphical user interface is represented on a first display of the housing of the attractor module;
the attractor module having an attractor display;
wherein the attractor display attracts a gaze of a patient;
wherein the gaze of the patient includes an accommodation;
wherein the attractor module minimizes the accommodation;
wherein the attractor module provides an optimal accommodation for achieving a best measurement;
the attractor module having a camera;
the attractor module having an onboard computing system;
the attractor module having a communications system;
wherein the camera captures a plurality of images of an eye of the patient;
wherein the plurality of images captured are used for vision screening.

2. The system of claim 1, further comprising:
the attractor display having a diffuser;
the attractor display having a plurality of lights;
wherein the diffuser and the plurality of lights attract the gaze of the patient.

3. The system of claim 1, further comprising:
a processor;
a non-transitory storage memory;
wherein the non-transitory storage memory is associated with the processor;
the non-transitory storage memory having a program code with a predetermined set of rules which execute a program;
wherein the predetermined set of rules display an image on the attractor display.

4. The system of claim 1, further comprising:
the attractor module having a diffuser liner;
the attractor module having a diffuser housing;
wherein the diffuser causes light of the plurality of lights to appear blurry.

5. The system of claim 1, further comprising:
the attractor display having a diffuser;
the attractor display having a plurality of lights;
wherein the diffuser and the plurality of lights attract the gaze of the patient;
wherein the plurality of lights are of a red light in order to preserve a desired pupil dilation;
wherein the red light is in the red or infrared region of the light spectrum.

6. The system of claim 1, further comprising:
the attractor module having a flash module.

7. The system of claim 1, further comprising:
automated examination;
wherein a processor carries out examination of the images based on a predetermined set of rules;
wherein the processor returns a diagnosis.

8. The system of claim 1, further comprising:
wherein the camera captures images of an eyeball of the patient;
wherein the camera captures images of a flash reflecting off a retina of the patient.

9. The system of claim 1, further comprising:
a processor;
a non-transitory storage memory;
wherein the non-transitory storage memory is associated with the processor;
the non-transitory storage memory having a program code with a predetermined set of rules which execute a program;
wherein the predetermined set of rules display an image on an attractor display;
wherein the image displayed on the attractor display is sufficiently blurry that the image causes the patient viewing the image to focus on the image as if the image were located at a further distance from the patient than the attractor display is located.

10. The system of claim 1, further comprising:
a mobile computer apparatus;
the mobile computer apparatus having a second display.

11. The system of claim 1, further comprising:
a predetermined set of rules;
wherein the predetermined set of rules can be changed to cause various images to appear on the attractor display.

12. The system of claim 1, further comprising:
the attractor module having a power supply.

13. The system of claim 1, further comprising:
the attractor module having a power supply;
wherein the power supply is a plurality of batteries.

14. The system of claim 1, further comprising:
the attractor module having an audio feature;
wherein the audio feature is used to cause attention of the patient to gaze.

15. The system of claim 1, further comprising:
the attractor module having a plurality of lights;
wherein the plurality of lights are configured behind the diffuser;
wherein the plurality of lights are arranged so as to attract a gaze of a patient;
the patient having an accommodation of a particular focal distance;
wherein the attractor module induces the patient to focus at infinity to control the accommodation of the patient;
wherein controlling the accommodation of the patient provides a best measurement;
wherein the best measurement is an accurate measurement which can be used in vision screening.

16. A vision screening system, comprising:
a smart device;
the smart device having a graphical user interface;
the smart device having an onboard computing system;
the smart device having a communications system;
an attractor module;
the attractor module extending a length from a first end to a second end;
the attractor module having a housing;
the attractor module having an attractor feature;
a camera;
a flash;
wherein the attractor feature attract the gaze of the patient;
wherein the gaze of the patient includes a desired focus;
wherein the gaze of the patient includes a desired direction;
wherein the camera captures images of the patient.

17. The system of claim 16, further comprising:
the attractor feature having a diffuser;
the attractor attractor having a plurality of lights;
wherein the plurality of lights are configured behind the diffuser;
wherein the plurality of lights are arranged so as to attract a gaze of a patient;
the patient having an accommodation of a particular focal distance;
wherein the attractor module induces the patient to focus at infinity to control the accommodation of the patient;
wherein controlling the accommodation of the patient provides a best measurement;
wherein the best measurement is an accurate measurement which can be used in vision screening.

18. The system of claim 16, further comprising:
a processor;
a non-transitory storage memory;
wherein the non-transitory storage memory is associated with the processor;
the non-transitory storage memory having a program code with a predetermined set of rules which execute a program;
wherein the predetermined set of rules display an image on an attractor display;
wherein the image displayed on the attractor display is sufficiently blurry that the image causes the patient viewing the image to focus on the image as if the image were located at a further distance from the patient than the attractor display is located.

19. The system of claim 1, further comprising:
wherein the smart device acts as a power supply for the attractor module;
wherein the smart device provides a computing processor for the attractor module.

20. A method of vision screening, comprising the steps:
providing an attractor module; the attractor module displaying a blurry image;
capturing a desired focus of a patient with the attractor module;
capturing a desired direction of viewing of the patient with the attractor module;
providing a camera;
capturing images with the camera of an eye of a patient;
screening the images captured of the eye of the patient for vision impairment;
screening the images captured of the eye of the patient for vision problems.
